(19) United States
(12) Patent Application Publication
(10) Pub. No.: US 2020/0080115 A1
(43) Pub. Date: Mar. 12, 2020

(54) Cannabinoid Production by Synthetic In Vivo Means

(71) Applicant: Biotic Sciences LLC, San Francisco, CA (US)

(72) Inventors: Anthony Clark, New York, NY (US); Jayaranjan Anthonypillai, San Francisco, CA (US)

(73) Assignee: Biotic Sciences LLC

(21) Appl. No.: 16/389,577

(22) Filed: Apr. 19, 2019

Related U.S. Application Data
(60) Provisional application No. 62/661,064, filed on Apr. 22, 2018.

(51) Int. Cl. C12P 7/42; C07K 14/415; C12N 1/18; C12N 9/10

(52) U.S. Cl. CPC C12P 7/42; C07K 14/415; C12N 1/18; C12Y 205/0101; C12Y 205/01102; C12Y 205/01029; C12N 9/1085

(57) ABSTRACT

In some aspects of the design, novel forms of geranylpyrophosphate:olivetolate geranyltransferase; of olivetol synthase or of geranyl pyrophosphate synthase; of geranylgeranyl pyrophosphate synthase; of olivetolic acid cyclase; and/or of olivetolic acid synthase for making large scale amounts of cannabinoids in cells, in vitro are presented.

### RELATED APPLICATION

This application claims the benefit under 35 USC 119 of U.S. Provisional Patent Application No. 62/661,064, filed Apr. 22, 2018, titled “Cannabinoid Production by Synthetic in vivo Means,” which is hereby incorporated herein by reference in its entirety.

### INCORPORATION-BY-REFERENCE OF SEQUENCE LISTING

This application includes a Sequence Listing, which has been electronically submitted in ASCII format, and which is hereby incorporated by reference herein in its entirety. The Sequence Listing in ASCII format is named “0001P_SequenceListingV2.txt,” was created on Oct. 17, 2019, and has a size of 29 kilobytes. No new matter is added.

### FIELD OF THE INVENTION

The design relates to novel molecules, which can be obtained from sources other than Cannabis, with enzymic activity; novel cells comprising same; novel cell lines comprising same; novel organisms comprising same; novel in vitro cannabinoid-producing materials and methods; and novel materials and methods for large scale production of cannabinoids.

### BACKGROUND OF THE INVENTION

Plants synthesize a variety of hydrocarbons composed of isoprene units (“Methods in Plant Biochemistry,” Dey & Harborne, eds., Academic, San Diego (1991) 7:519-536). Entities with lower chain lengths and varying numbers of cis and trans double bonds may be known as polyprenols, while some of those of longer chain length may be identified as rubbers (Dey & Harborne, 7:537-542). Synthesis of such hydrocarbons includes a number of pathway enzymes, such as, enzymes associated with synthesis of polyketides (PK) or of terpenoids, including synthases that form some of the starting materials, and prenyltransferases which catalyze sequential addition of hydrocarbon units to a starting material.

Some enzymes involved in polyketide or terpenoid metabolism, such as, synthases, are dimers, some are homodimers and some may be heterodimers. Monomeric enzymes can be about 400 amino acids in size and each monomer of a dimer can be about 400 amino acids in size.

For example, common PK synthases (PKS) include valerophenone synthase which catalyzes a first step in α-acid and β-acid production in hop; chalcone synthase (CS), which is involved in flavonoid biosynthesis, stilbene synthase, which among other products is involved in resveratrol production; squalene synthase which is involved in sterol production; and so on.

PKS' are more common in plants and some bacteria, it was believed animals do not have or do not express PKS activity. For example, Saccharomyces cerevisiae does not make monoterpenes; and microbes generally do not have or express geranyl diphosphate (GPP or geranyl pyrophosphate) synthase (GPPS). In some yeast, a GPPS activity resides in a farnesyl diphosphate (or farnesyl pyrophosphate) synthase (FPPS) where it has been suggested that GPP is a precursor of FPP and no more, for example, a donor molecule for synthesis of larger PK's. S. cerevisiae FPPS is the expression product of the ERG20 locus, and the polypeptide has a Lys residue at position 197, crucial to FPPS activity. Mutation of K197 can alter the ratio of GPP to FPP synthesized (Fischer et al., Biotech Bioeng 108(8)1883-1892, 2011).

Cannabinoids have origins in both polyketide (phenolic) and terpenoid metabolism and often are considered terpenophenolics or prenylated polyketides. Cannabinoid biosynthesis occurs primarily in trichome glands of female flowers. Cannabinoids are formed by an initial three-step biosynthetic process: polyketide formation, prenylation and cyclization.

The polyketide, olivetolic acid (OTA), is formed by an OTA synthase (OAS) or by coordinated, sequential action of an olivetol synthase (OS, also known as a tetraketide synthase (TS)); and an OTA cyclase (OAC), from starting materials, hexanoyl-CoA and malonyl-CoA.

The second enzymatic step is prenylation of OTA with the terpenoid precursor, GPP, to form cannabigerolic acid (CBGA) by geranylpyrophosphate:olivetolate geranyltransferase, GOT, Fellermeier & Zenk (infra). Oxidocyclase enzymes convert CBGA to, for example, Δ⁹-tetrahydrocannabinolic acid (THCA) or to cannabidiolic acid (CBDA).

Although genes encoding many of such enzymes are cloned, expression of those nucleic acids in heterologous host cells, that is, non-Cannabis cells, has been poor or unsuccessful, and certainly from the standpoint of producing commercially viable amounts of cannabinoids at tolerable cost.

Moreover, cannabinoids of current medical significance are synthesized in appreciable amounts by essentially only two species of plants, C. sativa and C. indica.

Hence, there is a need for and/or will be an advantage in producing new cannabinoid pathway enzymes and using same for producing commercial levels of cannabinoids in Cannabis and non-Cannabis cultured host cells.

The design is in the field of plant genetics and biochemistry. More specifically, the design relates, in part, to enzymes and polypeptides, for example, from non-Cannabis plants, that are used as is or are modified to produce cannabinoids.

### SUMMARY OF THE INVENTION

Those needs and advantages were attained in development of materials and methods for large scale production of cannabinoids. Novel enzymes, such as, chimeric enzymes, for example, from organisms other than Cannabis, that catalyze steps in a cannabinoid synthetic pathway, such as, synthesis of geranyl diphosphate (GPP), are described, as is use thereof in cells, tissues and organisms for large scale production of cannabinoids in cell culture; as well as for use in cell-free reactions for producing cannabinoids at scale.

Novel polypeptides with olivetolic acid synthase (OAS) activity; with olivetol synthase (OS) activity to produce olivetol (OL) or a related tetraketide (TK); with olivetolic acid cyclase (OAC) activity; with geranylpyrophosphate:olivetolate geranyltransferase (GOT) activity, with GPP synthase (GPPS) activity, with geranylgeranyl pyrophosphate (GGPP) synthase (GGPPS) activity, with farnesyl diphosphate (FPP) synthase (FPPS) activity and other isoprenoid synthases, where one or more of those polypeptides can be from one or more non-Cannabis sources are used in large scale settings to produce commercial amounts of cannabinoids.

Molecules of interest can be obtained from non-Cannabis species and comprise polypeptides that can be substituted in and used in the cannabinoid synthetic pathway. Humulus lupulus (hop) is the closest extant relative of the genus, Cannabis. However, polyketide synthases and prenyl transferases of hops are targeted to form bitter acids (Li et al., Plant Physiol 167:650-659, 2015). Nevertheless, many hop enzymes are not specific as to substrate and accept a range of reactants beyond that or those used in vivo or in planta (to produce bitter acids) and thus, can be used to produce cannabinoids. Hence, hop enzymes, as well as enzymes of species systematically related to Cannabis can be used to make cannabinoids.

However, the source of polypeptides for making cannabinoids is not limited to Cannabis or Humulus species. Polypeptides can be obtained from other plants, but also from animals, microbes and so on.

Polypeptides of interest from non-Cannabis sources can be modified to reduce substrate specificity and to alter product formed. For example, amino acids strategic in engaging and orienting substrates or reactants, or, for example, reducing specificity for a naturally occurring reactant in vivo or in planta, and directing that modified enzyme to accept and to catalyze reaction with, for example, OTA, GPP, OL and so on can be modified and are provided in the instant application. Modification also comprises enabling a nucleic acid encoding a polypeptide of interest to be expressed in a host cell of interest.

The common origin, related structure and related function of enzymes or polypeptides of interest enable exchange of portions of enzymes, domains of enzymes, chains of enzymes and so on between or among enzymes for altering normal function subverting a polypeptide to make a non-natural product, for example, for producing, for example, OTA or GPP. For example, a chimeric dimeric polyketide synthase (PKS) or dimeric synthase in the terpenoid universe can comprise one monomer from one species and the other monomer from another species.

In embodiments, a polypeptide, which directs a product produced by an enzyme, polypeptide or polypeptides, is combined with a heterologous or endogenous polypeptide or polypeptides, coercing, adjusting, directing and so on, that or those heterologous or endogenous polypeptide(s) to produce, for example, GPP.

Polypeptides with defined enzymic activities of interest, which may be isolated from natural sources or can be cloned, optionally, altered, and expressed, also enable in vitro production of cannabinoids as all of the starting materials are available commercially or can be synthesized.

Polypeptides, and nucleic acids encoding same, enable production of cannabinoids in cells of species other than C. sativa or C. indica, such as, insect cells, other plant cells, yeast cells, bacteria cells, fungal cells, mammal cells and so on.

Recombinant nucleic acid materials and methods enable specific siting, trafficking, directing and so on of expressed polypeptides to particular sites and compartments in a cell to enable, for example, access to larger amounts or pools, or higher concentration of reagents or starting materials; access to upstream and/or downstream enzymes of a pathway; and so on to facilitate production of a desired product, such as, a cannabinoid.

### DRAWINGS

The drawings refer to some embodiments of the design provided herein in which:

FIG. 1 illustrates an example embodiment of a flow diagram of creating a composition with multiple polypeptides.

FIG. 2 illustrates an example embodiment of a flow diagram of making a cannabinoid.

### DETAILED DESCRIPTION OF THE INVENTION

The design relates to materials and methods for large scale production of cannabinoids.

As used herein, unless the context clearly indicates to the contrary, words used in the singular include the plural, and words used in the plural include the singular. As used herein, unless the context clearly indicates to the contrary, the article, “the,” is not limiting. As used herein, unless the context clearly indicates to the contrary, the term, “include,” has the meaning, “include, but is/are not limited to,” or, “comprise.”

“About,” is an approximation relative to a certain value such that an amount or level of variability exists that is reflected, for example, in an error range or value, or a deviation that provides a range of acceptable or usable values about that certain value, such as, ±10%, where the limits of the range are 10% less than the certain value, including the certain value and 10% greater than the certain value. Hence, as used herein, by reciting about 50, it is understood that the value can range from 45 to 55. In embodiments, limits of the range are ±5%. A synonymous term includes, “essentially.”

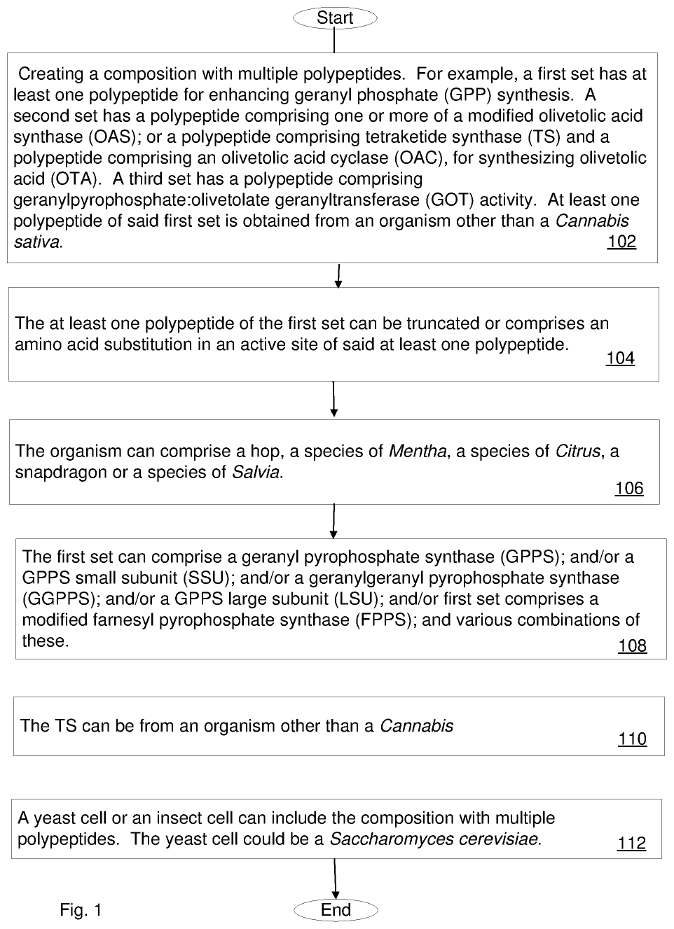

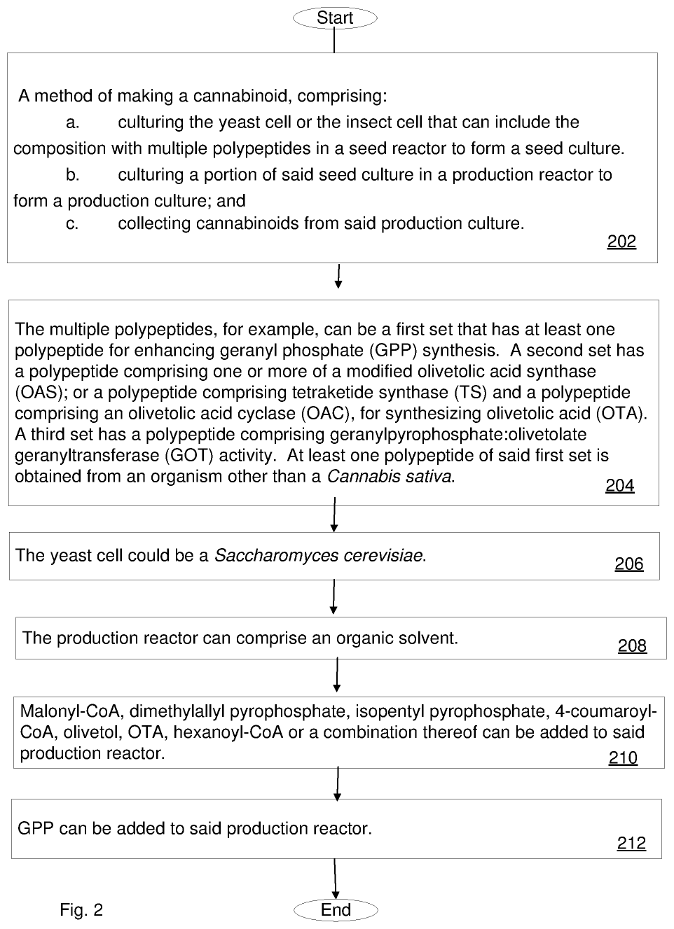

FIG. 1 (steps 100-112) and FIG. 2 (steps 200-212) refer to aspects of the design and will be further flushed out as discussed below.

As used herein, “an organism other than a Cannabis sativa,” means sourcing of a nucleic acid, a protein and so on from an organism this is not C. sativa. Equivalent forms of that phrase include, “is obtained from something, an organism, a species and the like that is not a Cannabis sativa”. For the purposes of the instant application, there is only one species of C. sativa that produces higher levels of cannabinoids, although there may be subspecies, varieties and so on that also produce cannabinoids, albeit at levels lower than found in wild type C. sativa. Hence, any molecule of interest that is not obtained from a C. sativa is one which is from an organism other than a C. sativa.

Olivetolic acid (OTA) synthase (OAS) is a polyketide (PK) synthase (PKS) that joins hexanoyl-CoA and three molecules of malonyl-CoA to form OTA. Plant PK's can share properties, such as, a Cys residue in the active site at about amino acid 164, as well as the Phe (215), His (303) and Asn (336) residues, also in the active site (numbering is of chalcone synthase (CS) of Brassica alba). Cannabis PKS proteins can have conserved amino acids, Cys 167, His 307 and Asn 340, which may occupy the active site (Jez et al., Biochem 39:890-902, 2000). Unlike CS, in an OAS of interest, Thr 300 may be replaced by Leu or Ile. The PKS of Raharjo et al. (infra) and of Marks et al. (infra) react with both hexanoyl-CoA and malonyl-CoA. Alteration of sequences, for example, amino acid substitution of the Raharjo et al. or of the Marks et al. enzyme and polypeptide can alter the product of the joining reaction.

OL is a decarboxylated OTA and is a diol. OS is a homodimer, and each monomer can comprise Cys 164, His 303 and Asn 336 residues in the active site. Hence, OS resembles CS. However, while CS has amino acids Thr 132, Thr 194 and Thr 197 in the active site (Jez et al., supra), OS has Ala, Met and Leu, respectively, at those sites, Taura et al. (infra). OS may produce OL or another product, such as, a linear tetraketide, since OL is not present in detectable amounts in C. sativa. OAC is related to dimeric α+β barrel proteins (Marks et al., infra and Gagne et al., infra) and cyclizes a linear TK to form OTA.

Chalcone synthase (CS) is a common enzyme across taxa. For example, CS is foundational in flavonoid biosynthesis. CS commonly is found in the cytoplasm. Three malonyl-CoA molecules and a 4-coumaroyl-CoA are joined to form naringenin chalcone. CS generally is a homodimeric protein with a monomer of about 45 kD in size.

FPP (farnesyl pyrophosphate) synthase (FPPS) generally is a homodimer. Some FFPS naturally form varying amounts of GPP. Some FPPS molecules have a critical amino acid, Lys197 that governs product specificity. Mutation of Lys to Glu results in that modified S. cerevisiae FPPS to make only GPP (Blanchard & Karst, Gene 125(2)185-189, 1993). Similarly, an avian FPPS mutant produces GPP (Fernandez et al. (also identified as Stanley-Fernandez et al.) Biochem 39(5)15316-15321, 2000).

GPPS is found commonly in microbes, plants and animals. GPPS can be a homodimer or a heterodimer with a large subunit (LSU) and a small subunit (SSU), for example, from a species of Lithospermum, a species of Salvia, such as, sage, a species of grape, a species of Arabidopsis, a species of Mentha, a species of Citrus, a species of cotton, a snapdragon, a hop, Clarkia breweri, a tobacco, Taxus canadensis, Abies grandis and so on. The subunits are about 400 amino acids. Some of the homodimeric enzymes contain characteristic DDXXD (SEQ ID NO:1) and FQXXDDXD (SEQ ID NO:2) (X is any amino acid) domains or motifs for binding substrate. Of heterodimeric forms, the SSU can be persuasive in directing or focusing catalytic activity, for example, to forming GPP or FPP.

GGPPS is a common enzyme and generally is a homodimer. GGPPS can be a promiscuous enzyme that produces not only GGPP but GPP as well. A monomer of the homodimer can have homology to GGPS-LSU. Some sources of GGPPS include Capsicum annuum, a species of Lupinus, an Arabidopsis, A. grandis, T. canadensis, a hop and so on.

GPP is a prenyl donor of OTA in the onset of cannabinoid biosynthesis. In planta, GPP is formed by condensation of dimethylallyl pyrophosphate (DMAPP, also known as dimethylallyl diphosphate) and isopentyl pyrophosphate (IPP, also known as isopentyl diphosphate) by a GPPS.

Prenylation is a common feature of Cannabis and the related species, hops. Li et al. (supra) cloned two prenyl transferases (PT), PTL1 and PT2, which catalyze successive reactions adding DMAPP to form bitter acids. A truncated PTL1 accepted instead GPP as a substrate. A wildtype PTL1 associated with a mutated PT2 containing amino acid substitutions in the PT2 active site, instead accepted GPP as a substrate. U.S. Pat. No. 8,618,355 also lists GPP as a prenyl donor of one of the two PT's disclosed therein actually tested, HIPT1. CsPT1 (U.S. Pat. No. 8,884,100) has homology to VTE2-2, homogentisate phytyltransferase, of tocopherol biosynthesis.

By, “optimized,” or grammatic forms thereof, is meant a polypeptide of interest, as well as a nucleic acid encoding that polypeptide, that is a changed or an altered form relative to the wild type polypeptide or polynucleotide and has properties or provides advantages over that of the wildtype polypeptide. The alteration can be one which provides a polypeptide that produces cannabinoids at a greater rate, a faster rate, a more efficient rate and so on than observed with wild type polypeptide; that operates in a particular non-Cannabis cell; the nucleic acid encoding same is cloned more efficiently in a host cell than other recombinant nucleic acids; the nucleic acid is expressed more efficiently or at greater levels in a host cell; and so on. The polypeptide can be a truncated form, a rearranged form containing multiple copies of a domain or rearranged domains; the nucleic acid encoding same can comprise codons optimized for the host cell, can comprise elements that enhance expression of the coding sequence and so on; a polypeptide of interest can comprise modified amino acids; and so on. Hence, optimized relates to any property of a polypeptide of interest, or a polynucleotide encoding same, that is higher, greater, better, faster and so on than wild type, that provides a benefit or advantage for making cannabinoids in cell culture.

By, “modified,” or grammatic forms thereof, is meant a nucleic acid and/or a polypeptide of interest that contains at least one change from the progenitor or wild-type or naturally occurring nucleic acid or polypeptide form from which the modified form is derived. Hence, for example, a nucleic acid encoding a polypeptide with an enzymic activity of interest can have codons optimized for a particular and foreign host cell; can be truncated; can comprise sequences adding one or more domains to the encoded polypeptide; can comprise a nucleic acid where encoded polypeptide domains are shuffled and not in a naturally occurring order; can comprise a foreign promoter, an enhancer and so on to enable expression in a foreign host; can comprise portions from a different gene, a different organism (a chimeric gene) can comprise alteration at one or more amino acids, such as, substitution, deletion, insertion or a modification, such as, a modified base or a modified residue; and so on. A modified polypeptide is one retaining an enzymic activity of interest but which may be truncated, may contain rearranged domains, may contain additional domains, may comprise an amino acid substitution, addition or deletion, may comprise a conservative amino acid substitution, may comprise an amino acid substitution that changes the structure and property of the replaced amino acid, may comprise an amino acid substitution that alters substrate specificity or product formed, may comprises an amino acid that is altered or derivatized, such as, carrying a new or different functional group, and so on.

“Isolated,” refers to a polypeptide or a nucleic acid that is removed from the native environment thereof, such as, a chromosome, a cell and so on.

Some embodiments of the design relate to an isolated or purified nucleic acid having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identity to a polypeptide or to a polynucleotide of interest. Degree of tolerable identity or homology is contingent on the expressed polypeptide having a substantial level of enzymic activity of interest.

In the context of catalytic or enzymic activity of a recombinant polypeptide of interest, “substantial,” and grammatic forms thereof, are meant that, relative to a particular catalytic metric of a wild type enzyme, a recombinant polypeptide with substantially the same catalytic activity of that wild type enzyme will have that particular metric at a level at least 80% of the value of that metric of the wild type enzyme. A level of catalytic activity less than 80% of that of the wild type enzyme can be acceptable if that recombinant has other beneficial and/or advantageous properties, such as, high level of expression, stability in a host cell, results in high levels of cannabinoid production and so on. In embodiments, substantial may relates to at least 90% of the metric.

As will be appreciated by a skilled practitioner, changes in nucleic acid sequence do not necessarily alter amino acid sequence of the encoded polypeptide. It will be appreciated that some organisms favor certain codons for maximal translation thereof. For example, yeast favor certain codons, JBC 257:3026 (1982). “Preferred yeast codons,” or, “yeast preferred codon,” are codons containing nucleotide bases that are observed and used more frequently than other possible codon triplets to encode particular amino acids in yeast, such as, S. cerevisiae. Ways in which the nucleotide sequence can be varied or shortened are known in the art, as are ways of testing the effectiveness of altered genes to express a polypeptide of interest in a particular host cell. It will be appreciated that degeneracy of codon usage can be practiced in the instant invention. All such variations of genes that express a polypeptide of interest therefore are included as part of the present disclosure.

Some embodiments relate to a vector, construct or expression system containing an isolated or purified polynucleotide having at least 80% sequence identity to a polynucleotide of interest. Accordingly, there is provided a method for preparing a vector, construct or expression system including such a nucleic acid, or a part thereof, in a sense or anti-sense orientation, or a complement thereof, for introduction into a cell.

In embodiments, an isolated nucleic acid, or vectors, constructs or expression systems comprising that isolated nucleic acid, may be used to create transgenic organisms. Therefore, an embodiment relates to transgenic organisms, cells or tissues of an organism including an isolated nucleic acid having at least 80% sequence identity to a polynucleotide of interest.

An organism can be a plant, a microorganism, a mammal or an insect, for example. Plants can be of the genus Cannabis, for example C. sativa, C. indica and C. ruderalis, but can include tobacco, rice, carrot, Arabidopsis and so on. Microorganisms can be bacteria (e.g. Escherichia coli) or yeast (e.g. S. cerevisiae, Schizosaccharomyces pombe, a Pichia and so on). Insect can be Spodoptera frugiperda cells or mosquito cells. Mammal can be human cells, such as, CHO cells, murine cells and so on.

Expression or over-expression of a nucleic acid may be in combination with expression or over-expression of one or more other nucleic acid(s) that encode(s) one or more enzyme(s) in a cannabinoid biosynthetic pathway, such as, nucleic acids that encode an OAS, an OS, a GPPS, a GGPPS, a CS, an FPPS, a GPPS-SSU, a GPPS-LSU, an OAC, a GOT, a tetrahydrocannabinolic acid (THCA) synthase, a cannabidiolic acid (CBDA) synthase, a cannabichromenic acid (CBCA) synthase and so on, or modified forms thereof.

As known, conservative substitutions of amino acids of a polypeptide can be tolerated without substantial disruption of polypeptide structure and/or function. Conservative substitutions can be made by substituting an amino acid with another of similar hydrophobicity, polarity, R group shape and/or property, and so on. Comparison of aligned sequences of homologous proteins of different organisms can reveal conservative substitutions that do not or minimally alter function of the proteins.

As known in the art, molecules of similar structure and/or function that arise from different genetic origin (that is, from different species, which nevertheless may share a recent common genetic origin or ancestor (monophyly or members of a clade) and hence, are plants or animals of recent speciation events) are identified herein as, “orthologs,” or “orthologues,” and grammatic forms thereof. Often, orthologs arise or exist because of speciation. Thus, related species that have a common genetic origin can contribute orthologs which may be interchangeable or may be modified for a particular function with only a small number of modifications. Sometimes, however, molecules of similar function can arise from convergent evolutional processes.

The phrase, “degree or percentage of sequence homology,” refers to level of identity between two sequences after maximal identity or matching alignment. Percent identity can be determined by comparing two maximally aligned sequences, where a portion of one of the polypeptides or one of the polynucleotides may comprise additions and/or deletions as compared to the other polypeptide or polynucleotide being compared to attain the greatest number of residues or bases matched in the two polypeptides being compared or in the two polynucleotides being compared. The percentage is calculated by determining the number of positions of matched amino acid residues or nucleic acid bases in both sequences, dividing that number by the total number of positions and multiplying the result by 100.

“Homologous sequence,” is understood to mean a sequence having a percentage identity of bases or amino acid residues of at least about 80%, at least about 85%, 86%, 87%, 88%, 89%, 90%, 91%, 92%, 93%, 94%, 95%, 96%, 97%, 98%, 99%, 99.5%, 99.6%, or 99.7% with a second sequence. Any differences between the two sequences can be random and/or over the length of the sequences. Sequence identity can be determined manually, using a computer program designed to perform single and multiple sequence alignments and so on.

As to, “sequence identity,” two polypeptides or two nucleic acids are said to be, “identical,” or to have identical sequence if the primary structure of amino acids of the two polypeptides or base sequence of nucleotides of the two nucleic acids is the same. Optimal alignment of sequences for comparison may be conducted by known homology algorithms (for example, as disclosed in Ad App Math 2:482 (1981); JMB 48:443 (1970); and PNAS 85:2444 (1988)) and available programs, such as, FASTA, BLAST (JMB 215:403-410, 1990) and so on.

As used herein, “substantially similar,” refers to comparison of two entities where the difference of a metric of the two entities is no greater than 20%. Hence, if the difference of a metric of two entities is less than 20%, for purposes herein, the two entities are the same as to that metric. It is therefore understood that the invention encompasses more than the specific exemplary sequences.

For example, in embodiments, similarity of nucleic acids (that is, complementarity) can be ascertained by sequencing but also by determining propensity of single strands of nucleic acids to Watson-Crick hybridize and to anneal to form double stranded hybrids, and the stability thereof, for example, under stringent conditions (for example, 0.1×SSC, 0.1% SDS, 65° C.).

“Altered levels,” relate to a metric, such as, production of gene product(s) in cells or organisms, where the metric is present in an amount or a proportion that differs from that metric of normal, pretreatment, wildtype, non-transformed and so on, cells or organisms, essentially, a positive control or any selected reference entity. That altered metric value can be higher or lower, larger or smaller, greater or lesser and so on, than the metric value of the positive control or of the reference entity, such as, a wild type form.

A, “mature,” protein is one that is a post-translationally processed polypeptide; i.e., one from which any prepeptides, propeptides and so on, present in the primary translation product, are removed. A, “precursor,” protein refers to the primary product of translation of mRNA; i.e., with prepeptides, propeptides and so on, still present. Prepeptides, propeptides and so on may be but are not limited to intracellular localization signals.

A, “recombinant,” molecule, such as, a polypeptide, is one which is not naturally occurring or is made by artificial combination of two otherwise separated segments of molecules by, for example, chemical synthesis, or artificial manipulation of isolated segments of molecules, e.g., by genetic engineering techniques, and so on. Thus, a recombinant nucleic acid or polynucleotide as used herein relates to a polynucleotide of genomic, cDNA, semisynthetic or synthetic origin which, by virtue of origin or manipulation: (1) is not associated with all or a portion of a nucleic acid associated therewith in nature, (2) is linked to a nucleic acid other than that to which the nucleic acid is linked in nature, or (3) does not occur in nature. As used herein, the translation product of a recombinant nucleic acid of interest can be identified as a recombinant protein or a recombinant polypeptide.

“Fragment,” refers to a nucleic acid or a polypeptide comprising a portion of a larger nucleic acid or polypeptide of the design. An, “active,” fragment comprises a sufficient portion or quantity of desired activity or function. A nucleic acid fragment may consist of a significant portion of or most of the nucleic acids of the design. Producing a recombinant polypeptide with the requisite catalytic activity is determinative. Similarly, a polypeptide can be truncated so long as the remaining fragment comprises a substantial level of the requisite catalytic activity of the parent wild type enzyme, at least about 80% of activity of the intact polypeptide, at least about 85% of the activity of the intact polypeptide, or more.

“Host cell,” and, “host organism,” refer to a cell or to an organism that can receive or received a foreign or heterologous nucleic acid of interest and expresses same. Suitable host cells include microorganisms, such as, bacteria and fungi, as well as plant and animal cells. Many plants are suitable hosts for transforming with foreign genes including commercially important crops, such as, rapeseed, sunflower, rice and soybean. Suitable yeast cells include S. cerevisiae and Pichia pastoris. Host cells include progeny of a single host cell. Progeny cells may not be identical (in morphology, in the genome and so on) to the original parent cell due to natural, accidental or deliberate mutation but remain of interest as having a substantial level of a function of interest.

Choice of host may dictate choice of other reagents, such as, control sequences of a coding nucleic acid associated with an expression system as well as selectable marker.

As used herein, a, “vector,” refers to a nucleic acid that transports another nucleic acid linked thereto and can include a plasmid, a cosmid, a bacmid, an artificial chromosome (AC), such as, binary AC's, yeast (Y) AC's and so on, a virus or part thereof, vehicles made from a virus and so on. Vectors can function to insert a polynucleotide into a cell, to replicate a polynucleotide, to express a polynucleotide, to insert a polynucleotide in a genome; and so on, or combinations thereof. A vector can replicate autonomously in a cell or can integrate into a host genome.

“Synthetic,” or grammatic forms thereof, indicate artificial or non-naturally occurring. Synthetic can mean created de novo using individual reactants, building blocks and so on, or can relate to segments, fragment or portions that are pieced together to form a chimeric entity, and so on.

“Synthetic gene,” or, “synthetic nucleic acid,” can be assembled from manipulated nucleic acids or from oligonucleotide building blocks, that can be synthesized chemically using procedures known to those skilled in the art, such as, using an oligonucleotide synthesizer; or can be portions of naturally occurring polynucleotides that are pieced or joined together. Building blocks can be segments ligated using an enzyme to form gene segments which then can be assembled to construct an entire gene.

“Synthetic protein,” or “synthetic polypeptide,” relates to one that does not occur naturally, and may be, for example, constructed by chemical reaction from amino acids, chemical synthesis of oligopeptides or polypeptide fragments (such as, a conjugated protein), created by joining polypeptide in vivo (such as, a fusion protein), chemical or enzymatic modification of an amino acid in a polypeptide; or can be potions of naturally occurring polypeptides that are pieced or joined together; and so on.

“Chemically synthesized,” as relating to a nucleic acid, means that the component bases or nucleotides are, or the final construct is assembled by a chemical reaction, for example, in vitro. Manual chemical synthesis of a nucleic acid may be by using well established procedures or automated chemical synthesis can use any of a number of commercially available devices. With reference to a polypeptide, amino acids can be joined by chemical reaction, such as, an esterification or condensation reaction.

A, “foreign,” nucleic acid or polypeptide refers to a nucleic acid or a polypeptide not normally found in a host organism, but which is introduced into that host organism by, for example, gene transfer, such as, by a transformation procedure, such as, using biolistic particles, exposure to certain salts, exposure to an electric field, treated with an enzyme, for example, to form spheroplasts, using vectors of cancer origin or are cancer forming and so on. Foreign nucleic acids can comprise native nucleic acids inserted into a non-native organism, or chimeric nucleic acids. Generally, any nucleic acid introduced into a cell is foreign, even if obtained from the very cell in which the removed nucleic acid is introduced.

Foreign also can refer to an entity that is of origin different from that of a reference entity. For example, in a protein of quaternary structure having three polypeptides or monomers, A, B and C, in a chimeric form of that protein, polypeptide B may or may not be derived from an originating organism, X, but if B is designated the reference, then A and C are foreign to B. A and C can be obtained from the same or different organisms. If A, B and C are from the same organism and B is replaced by a B from another organism, then B is foreign to A and C.

“Chimeric,” or grammatic forms thereof, as relating to a nucleic acid, refers to a non-native nucleic acid, comprising, for example, regulatory and coding sequences that are not found together in nature, often termed a, “construct.” Hence, portions of a construct can arise from different genes, different organisms and so on. Accordingly, a chimeric gene is a synthetic gene. (A polypeptide, cell, organism and so on can be chimeric or a chimera, that is, composed of two or more genetic lineages.)

A, “transgene,” is a nucleic acid, such as, a foreign nucleic acid, that has been introduced into a host cell, for example, by a transformation procedure.

Although conventional sugars and bases can be used in practicing a method of the invention, substitution of analogous forms of sugars, purines and pyrimidines can be advantageous in designing a final product, as can alternative backbone structures like a polyamide backbone in a nucleic acid. Similarly, a polypeptide may comprise modified amino acids and may be interrupted by non-amino acids. A polypeptide may be modified naturally or by intervention; for example, disulfide bond formation, glycosylation, lipidation, acetylation, carboxylation, phosphorylation, ubiquitination, pegylation or any other manipulation or modification. Also included are polypeptides containing one or more analogs of an amino acid, see, e.g., Sambrook, et al. (infra) and Current Protocols in Molecular Biology, Ausubel et al., ed., Greene Publishing and Wiley-Interscience: New York (1987).

“Promoter,” refers to a nucleic acid that controls expression of a coding sequence or a functional RNA. In general, a coding sequence is located downstream of a promoter. A promoter can comprise proximal and more distal upstream elements, the latter elements often referred to as enhancers. Accordingly, an, “enhancer,” is a nucleic acid which stimulates promoter activity and may be an innate element of a promoter or a heterologous element inserted to enhance activity of or tissue-specificity of a promoter. A promoter often include, “a transcription initiation region,” which often includes, “an RNA polymerase binding site,” (for example, a “TATA box”) and, “a transcription initiation site.” A promoter herein also may have a second domain called, “an upstream activator sequence,” (UAS) that, if present, generally is distal to the structural gene (i.e., further upstream) relative to the transcription initiation region. The UAS also governs regulation of expression. Regulated expression may be either positive or negative, thereby either enhancing or reducing transcription, as desired. A promoter that is not regulated and essentially enables a tonic level of transcription of the open reading frame which is controlled by that promoter is known commonly as a constitutive promoter. A promoter may be activated by a stimulus thereby causing transcription of the controlled open reading frame and such a promoter is known commonly as an inducible promoter.

A, “translation leader sequence,” refers to a nucleic acid generally located between a promoter and the coding sequence. A translation leader sequence can be present in a fully processed mRNA, for example, upstream of a translation start sequence. A translation leader sequence may impact processing of a primary transcript to an mRNA, mRNA stability or translation efficiency. Examples of translation leader sequences can be found in Mol Biotech 3:225 (1995).

A, “3′ non-coding sequence,” refers to a nucleic acid located downstream of a coding sequence and can include, “a polyadenylation (poly-A) recognition sequence,” and other sequences encoding regulatory signals that impact mRNA processing or gene expression. A polyadenylation signal usually results in addition of a polyadenylic acid tract to the 3′ end of an mRNA precursor. Use of 3′ non-coding sequences is exemplified in Plant Cell 1:671-680 (1989).

The term, “operably linked,” or, “linked operably,” refers to association of nucleic acids pieces or fragments on or into a single nucleic acid so that function of one fragment impacts function of another. For example, a promoter is operably linked with a coding sequence when the promoter impacts transcription of that coding sequence (i.e., the coding sequence is under transcriptional control of the promoter). Coding sequences can be linked operably to regulatory sequences in sense or antisense orientation.

A, “chloroplast or plastid transit peptide,” is translated with a protein and directs a protein to a chloroplast or other plastid in a cell in which the protein is made. “Chloroplast transit sequence,” refers to a nucleic acid that encodes a chloroplast transit peptide.

A, “signal peptide,” is translated with a protein and directs the protein to the secretory system of a cell. If a protein is to be directed to a vacuole, “a vacuolar targeting signal,” can be added to the expressed polypeptide, or if to be directed to the endoplasmic reticulum, “an endoplasmic reticulum retention signal,” may be added. If a protein is to be directed to the nucleus, any signal peptide should be removed and instead, “a nuclear localization signal,” included. A signal peptide (i.e., a “signal sequence” or “leader sequence”) can be derived from a yeast gene coding for a polypeptide that is secreted, such as, signal and prepro sequences of yeast invertase (SUC2) and α-factor, repressible acid phosphatase (PHO5) genes and so on, the glucoamylase signal sequence from Aspergillus awamori and so on.

Such transit peptides and other peptides that site, direct, target, move and so on a polypeptide to a particular compartment, organelle, site and the like of a cell enable manipulation of expressed polypeptide of interest to advantage to a particular site, organelle and so on of a cell where reagents are found or are in higher concentration, where pathway enzymes are sited and so on for enhanced expression of a polypeptide of interest, and hence, of cannabinoids.

In embodiments, a gene product is released and operates in the cytoplasm and may be independent of, for example, the Golgi apparatus or the endoplasmic reticulum, and is not contained in an organelle, such as, a mitochondrion or a plastid. In those situations, a translated polypeptide may not contain a transit, targeting or leader sequence or polypeptide.

“Transformation,” refers to transfer of a foreign nucleic acid into a host cell using any method, including, for example, calcium phosphate or calcium chloride coprecipitation, lithium acetate transformation, DEAE-dextran-mediated transfection, lipofection, electroporation and so on. Host organisms containing a transformed nucleic acid fragments may be referred to as, “transgenic,” organisms. Examples of methods of plant transformation include Agrobacterium-mediated transformation and particle accelerated or, “gene gun,” technology. Transformation may be transient or stable.

A, “termination regulatory region,” may be derived from the 3′ region of the gene from which the promoter was obtained or from another gene. Suitable termination regions are known and include Agrobacterium tumefaciens nopaline synthase terminator (Tnos), A. tumefaciens mannopine synthase terminator (Tmas), the CaMV 35S terminator (T35S), the pea ribulose bisphosphate carboxylase small subunit termination region (TrbcS) and so on. Such gene constructs may be screened for activity by transformation into a host plant via Agrobacterium and screening for altered cannabinoid levels.

Selectable markers suitable for use in bacteria, such as, E. coli, an Actinomycetes and so on, generally impart antibiotic resistance; and those used in yeast often complement nutritional requirements. Selectable markers for use in yeast include, but are not restricted to, genes, such as, URA3, LEU2-d, TRP1, LYS2, HIS1 and HIS3. Selectable markers for use in Actinomycetes include, but are not restricted to those for thiostrepton resistance, apramycin resistance, hygromycin resistance and erythromycin resistance. Selectable markers not only enable transformed entities to survive selection, but also enable identification of transformants, selection and purification of transformants and so on.

A host cell can be treated to contain more than one foreign nucleic acid and thus, can be exposed to multiple gene transfer procedures.

One or more expression systems introduced into a host may be integrated into the host genome. Integration into the genome can increase stability of the heterologous genes over successive generations. Furthermore, subsequent procedures with the transformant can be easier to realize without concern of loss of or alteration of a vector during maintenance and processing of the transformed host cell. For example, homologous recombination can be used relying on adding sequences hybridizable to a host genome site to insert a transgene at that targeted site in the host genome (DeMarini et al., 2001, BioTechniques 30:520-52).

Practice of the design employs, unless otherwise indicated, conventional techniques of molecular biology (including recombinant DNA techniques), microbiology, cell biology, biochemistry, tissue culture, animal husbandry, agriculture, laboratory animal practice, immunology and so on, which are within the skill of the art. Such techniques are explained fully in the literature, such as, Sambrook et al., Molecular Cloning: A Laboratory Manual; CSHL Press: Cold Spring Harbor, 1989; “Oligonucleotide Synthesis” (Gait, ed., IRL Press, Oxford, 1984); “Culture of Animal Cells” (Freshney, ed., John Wiley, 2011); “Gene Transfer Vectors for Mammalian Cells” (Miller & Calos, eds., CSHL, NY, 1987); “The Polymerase Chain Reaction” (Mullis et al., eds., Birkhauser, 1994); “Yeast Genetic Engineering” (Barr et al., eds., Butterworths, Boston, 1989); and so on, or as known in the art.

Nucleic acid isolation and cloning are known, and, similarly, an isolated nucleic acid may be inserted into a vector and transformed into a cell by conventional techniques. There are a number of ways by which nucleic acids, vectors, constructs and expression systems can be introduced into organisms, and a combination of transformation and tissue culture techniques are used to create transgenic cells and organisms.

Recombinant expression vectors of the invention can be designed for expression of a transgene of the invention in non-Cannabis cells, such as, insect cells and yeast cells. Methods of expressing proteins in yeast, such as S. cerevisiae, P. pastoris, Hansenula polymorpha, Kluyveromyces lactis and so on are known in the art.

Thus, to produce cannabinoids of the invention, suitable hosts are modified to contain vectors, such as, plasmids, which contain expression systems for production of enzymes or polypeptides with the enzymic activity(ies) of interest. By placing polypeptides with separate enzyme activities on different expression vectors, variation can be achieved. Suitable host cells include yeast, E. coli, Actinomycetes and insect cells, such as, S. cerevisiae, P. pastoris, various strains of Streptomyces and so on. Expression of the enzyme activity(ies) of interest enable the transformed host cells to produce cannabinoids or to produce cannabinoids better.

As cannabinoid synthesis occurs primarily in plants (although the following discussion focuses on systems usable with plant cells and yeast cells, materials and methods for using other host cells are available and can be used herein), vectors for use in plant cells and in plants are known and include materials and methods for Agrobacterium-mediated transformation by vacuum infiltration or by wound inoculation; Agrobacterium Ti plasmid-mediated transformation of hypocotyl or cotyledonary petiole, by a wound infection method; particle bombardment/biolistic methods; polyethylene glycol-assisted gene transfer, protoplast transformation and so on, see, for example, Plant Molecular Biology: A Laboratory Manual, Clark, ed., Springer, N.Y. (1997).

Promoters for use in plant cells include, such as, constitutive promoters, inducible promoters, tissue-specific promoters and so on, and combinations thereof. Useful promoters include, but are not limited to promoters, such as, that of carnation etched ring virus (CERV), of cauliflower mosaic virus (CaMV) (19S or 35S promoter), a double enhanced CaMV promoter comprising two CaMV 35S promoters in tandem (referred to as, “Double 35S,” promoter), a NOS, OCS, RuBISCO or a napin promoter (napin for expression of transgenes in developing seed cotyledons); promoters for synthesis of glycolytic enzymes, such as, 3-phosphoglycerate kinase; promoters for alcohol dehydrogenase (ADH-1 and ADH-2), isocytochrome C or acid phosphatase, sucrose synthase and so on, promoters for degradative enzymes associated with nitrogen metabolism; promoters for enzymes responsible for maltose and galactose utilization; promoters associated with CYC1, HIS3, GAL1, GAL10, PHO5, GAPDH, ADC1, TRP1, URA3, LEU2, ADH, GS2, CHS, PAL, ENO, TPI (useful for expression in Saccharomyces) and so on, as well as other promoters. In the case of Pichia, an example is the AOX1 promoter. While many of the embodiments exemplify plant systems, other host cells can be used to advantage, for example bacterial cells for shuttle vectors. Suitable promoters for bacterial systems include lac, trp, IP_{L}, IP_{R}, T7, tac and trc, useful, for example, in E. coli.

A vector or construct may also include other elements conducive to expression in a plant cell, such as, an Adh intron 1, a sucrose synthase intron, a TMV Ω element and so on.

The invention provides, in part, a yeast-based system for requisite enzymic activity and cannabinoid production. Yeast are transformed with three or more vectors (each vector comprising a set to endow a host cell with a function, such as, enhanced GPP production, production of OTA and GOT activity), where each of the three or more vectors can be an episomal expression plasmid, and can include a yeast promoter and terminator, a selection gene, a nucleic acid for the enzyme activity of interest, optionally containing yeast preferred codons, and an origin of replication, such as provided by the 2μ sequence of yeast plasmid.

The yeast promoter may be a regulated promoter; generally the terminator will match the promoter. In embodiments, a vector may contain a yeast signal (leader) sequence for secretion extracellularly, however, in embodiments, a polypeptide of interest does not comprise a leader sequence as that polypeptide is destined to be maintained intracellularly to interact with other reagents in the cytoplasm, a plastid, an organelle and so on to form cannabinoids as desired.

In embodiments, a vector may be a shuttle vector which can function in plural hosts, for example, to facilitate construction thereof, amplification thereof and so on in a, for example, bacterium, prior to introduction into a yeast production host cell.

In embodiments, a vector may be an integrating vector which can deliver a nucleic acid into a host genome, which can be integrated in a unique and site-specific manner, for example, to facilitate expression thereof, to enhance stability thereof of a gene in a yeast production host cell and so on.

Transformed yeast may be selected by growth on a medium appropriate for a selectable marker of a vector, for example, a uracil deficient medium.

In fermentation, if a regulatable promoter is used, the promoter can repress recombinant polypeptide synthesis, allowing biomass to accumulate, followed by polypeptide production when the promoter is derepressed. Such a fed batch fermentation process allows for early dense culture development with increased cell mass while maintaining aerobic conditions and eventual high levels of cannabinoid production.

The production method of interest produces strikingly high yields of recombinant polypeptide and cannabinoid in yeast; e.g., levels in a culture medium can be between about 0.75 and about 5 g/L, as known in the art, yields also may be expressed as g product/g wet weight of biomass, g product/g dry weight of biomass, or g product/OD unit.

Ergosterol often is important for growth of certain microorganisms, such as, fungi, such as, certain yeast. To draw carbon metabolism to production of a recombinant polypeptide of interest and of cannabinoids, ergosterol production by a host cell can be reduced, for example, by replacing promoters of ergosterol biosynthesis with promoters which may be weakly constitutive and/or repressible. That strategy can be used with other genes, the expression of which can be lowered without detriment to the host cell. For example, a native promoter of a squalene synthase encoding gene, ERG9, in yeast can be replaced by a weakly constitutive or a repressible promoter, such as, an MET3 promoter.

In embodiments, a, “gene editing,” method can be used to disable, to replace and so on, an endogenous coding sequence. For example, a meganuclease, a zinc finger nuclease, a transcription activator-like effector-based nuclease, a clustered regularly interspersed short palindromic repeats system and so on can be used to remove, to inactivate, to modify, to suppress, partially or completely and so on, a particular sequence encoding an enzyme necessary, for example, in ergosterol synthesis.

Commercially available gene editing kits can be employed.

Hence, in embodiments, a coding sequence of interest can be replaced, inactivated, suppressed and the like, if amendable, by a gene editing technique to obtain, in part, a host cell that is manipulated to express cannabinoids.

Selected transformants may be plated on selection medium. e.g., if a URA3 gene is used as a selection gene, transformants may be plated on uracil minus plates (Ura⁻ plates) and incubated until viable colonies are visible. Individual transformants may be streaked onto, e.g., Ura⁻ plates, and colonies used to inoculate cultures under selection conditions, e.g., Ura⁻/6% glucose cultures. Transformants may be inoculated into a non-selective medium, such as, Yeast Extract Peptone Dextrose (YEPD), for shake flask culture. Cultures may be monitored for growth and analyzed for expression and other characteristics (e.g., level or amount) of a desired polypeptide. A clone with optimal combination of expression of a functional recombinant polypeptide with the requisite enzymic activity and other desired characteristics may be selected and a glycerol stock prepared for storage in a cell bank.

Processes for preparation of glycerol stocks are known. An exemplary process is to grow cells to an optical density (OD) of about 4, then to add sterile glycerol to a final concentration of about 20%, and then to freeze the cells in vials at −80° C. Cells may be stored at −80° C. or colder.

Generally, transformed yeast is used according to methods of the invention to produce a desired polypeptide by culturing the yeast. “Culturing,” in the context of the design has the usual meaning, i.e., growing cells, for example, that express a desired transgene. Specific culture conditions will depend on the cells used, polypeptide produced and the cannabinoid(s) produced. Cells comprising or contained in a vessel, such as, a flask or a fermenter, wherein the vessel is culturing the cells, the liquid mixture in that vessel often and can be termed, “a culture,” or a, “culture of cells.” Hence, cells of or the mixture in a seed fermenter can be called a, “seed culture,” cells of or the mixture in a production fermenter can be called, “a production culture,” and so on.

Cannabinoids may accumulate in cells. Cannabinoids may reside in the cytoplasm; may be synthesized in an organelle, such as, a chloroplast, a mitochondrion and so on; may be stored in a vacuole; and so on. Some cannabinoids are lipophilic and may accumulate in the plasma membrane in which case, a suitable surfactant can be included during lysis. Cannabinoids may be released into the medium. Hence, medium is processed for cannabinoids as well.

For production, an inoculum is used for initiating fermentation in a shake flask, the shake flask is used to start seed fermentation for increasing biomass forming a seed culture, and finally a production fermenter is used to form a production culture from a portion of a seed culture to maximize expression and yield of recombinant polypeptide(s) and hence, of cannabinoid(s). Glucose concentration can be regulated as described herein to maximize production. On conclusion of fermentation, cells may be separated from medium, lysed and the lysate clarified. Cannabinoids may be extracted from the medium as well. In embodiments of the invention, any of various methods may be used to purify cannabinoids to a desired level of purity.

The process can be divided into phases. In a batch phase (usually performed in inoculum shake flasks and/or seed fermenters), cells start growing to form a seed culture. In a fed batch phase (which generally occurs in a main or production fermenter or reactor), cells continue to grow at a controlled rate determined by feed rate to form a production culture.

In a continuous phase or in continuous culturing, a reactor is likened to a cell settler and runs continuously with dead cells and cannabinoids removed from the medium and dead cells, while viable cells continue to respire and to express transgene, and hence, to produce cannabinoids. Continuous culture enables a production culture to run for extended periods thereby increasing yield and efficiency as waste product and cannabinoids are removed continuously from a production fermenter and nutrients replenished continuously by feed introduction.

In embodiments where a promoter is used that is repressible, for example, by high levels of a carbon source (e.g., the ADH2 promoter is repressed by glucose), expression of a desired polypeptide is delayed until fed batch phase, when carbon source levels drop below levels that repress a promoter.

Dissolved oxygen, pH, ethanol concentration, carbon source (e.g., glucose) concentration, temperature, culture biomass, growth rate and the like are measured by standard instrumentation. Monitoring changes in one or more of those parameters provides feedback for maintaining proper feed rate to attain maximal environment conditions for maximal cell viability, which allows buildup of biomass while maintaining specific productivity of a transgene(s) in culture.

In a fed batch phase, feed rate of a carbon source (e.g., glucose) can be adjusted by controlling feed concentration and/or rate so that cells continually deplete the carbon source, while oxygen levels remain high, so that the cells are maintained in a respiratory state, rather than a fermentative state, thereby achieving optimum polypeptide production.

As used herein, “feed rate,” or, “rate of feeding,” refers to rate at which glucose or other carbon source is introduced into a culture medium, and may be altered by adjusting concentration and/or introduction rate of feed. Methods of monitoring state of a culture to determine if cells are in the respiratory state include (in addition to those disclosed above or known in the art) measurements of CO₂ evolution and O₂ uptake, ratio of those two metrics is expressed as respiratory quotient (RQ). In addition, dissolved oxygen is present continually during fed batch phase. Level of dissolved oxygen in the culture medium may be from, e.g., a minimum of about 30% to about 100% saturation, or greater than 50% saturation.

In a continuous phase, feed rate of the carbon source (e.g., glucose) also can be adjusted by controlling feed concentration and/or rate and dissolved oxygen and is maintained at an optimum level so cells continually dilute fermenter or reactor contents and are maintained in respiratory state rather than fermentative state thereby forcing and achieving optimal polypeptide production. Level of dissolved oxygen in the culture medium may be from, e.g., a minimum of about 10% to about 100% saturation, or greater than 50% saturation. For longer runs, OD at 600 nanometers (nm) (OD₆₀₀) can be used to monitor cell density within a reactor. Measureable OD₆₀₀ may be between 1 and 100, or greater than a 100.

For larger production runs, fermentation can be scaled in stages where size of fermentation vessel is increased incrementally. Normally, fed batch phase does not commence until fermentation is occurring in the largest vessel. Thus, for example, in larger production runs, production may start with shake flasks, for example, 2.8 L flasks containing about 600 mL of medium, which are seeded with recombinant yeast, which can be from a frozen cell bank. After cell density in the shake flasks reaches an optimum level (e.g., an OD₆₀₀ of between about 2 to about 10, or about 2.5 to about 9), contents of the shake flask are transferred to a seed fermenter. A seed fermenter may have a size between, e.g., 20 to 30 L, and contains between, e.g., about 10 and about 20 L of appropriate medium. After cell density in the seed fermenter reaches an optimum level (e.g., an OD of between 3 and 15), a portion of the contents of the seed fermenter optionally then is transferred to a secondary seed fermenter. A secondary seed fermenter may have a size between, e.g., about 100 to about 200 L, and contains between, e.g., about 65 and about 140 L of appropriate medium. After the cell density in the secondary seed fermenter reaches an optimum level (e.g., an OD of between 3 and 15), a portion of the contents of the secondary seed fermenter then is transferred to the main fermenter, which may have a size between, e.g., about 1000 L to about 2000 L, and which may contain between, e.g., about 500 and about 1000 L of medium. Fermentation is terminated in the main fermenter when extracted levels of cannabinoids achieve a desired level over a desired time period.

During continuous phase, which can be determined experimentally as the time point at which the highest process productivity is reached, for example (cannabinoid amount per fermenter volume per process time), cannabinoids are isolated continuously by conventional means known in the art. For example, a first step can consist of lysing cells and removing cells and cell debris by centrifugation. Methods for isolating, purifying and characterizing cannabinoids are known. Portions of medium also can be removed to obtain cannabinoids with replenishment of culture medium to maintain a steady state environment and culture in a reactor.

In embodiments, a medium is selected for growth of a transformed host cell and that can be different from that used for production of recombinant polypeptide and cannabinoids from that host cell. The latter can be favored by bringing a host cell to a life cycle stage where replication is minimized and transcription and/or polypeptide synthesis is maximized. Hence, cultivating a host cell and/or isolating cannabinoids from a medium and cell lysate can be performed via a multiphase or a continuous fermentation.

Immiscible and biocompatible organic solvents can be added to a production or to a cultivation medium to facilitate product collection.

Suitable organic solvents form a second, preferably hydrophobic phase, that can accumulate cannabinoids and/or avoid loss thereof through evaporation or other phenomena. Preferably, organic solvent does not impact significantly growth and/or survival of the host cell. Suitable solvents include diisononyl phthalate, dibutyl phthalate, oleyl alcohol, dihexyl ether and dodecane, for example.

While the above discussion describes a yeast system where expressed nucleic acids of interest are cloned in vectors suitable for a yeast host, and yeast cells are manipulated as taught herein, propagated and grown under large scale conditions to produce cannabinoids for extraction, purification and use, other host cells can be used in place of yeast cells, and in place of S. cerevisiae cells, such as, another yeast, a bacterium, a plant cell, a mammal cell, an insect cell and so on, adapting reagents for compatibility with a selected host cell, and culturing a host cell under conditions which favor cell growth and proliferation, as well as, recombinant polypeptide expression and cannabinoid production in that selected host cell.

The invention relates in part to expression of polypeptides that produce or facilitate GPP production; of polypeptides that comprise OAS, or OS/TS and OAC; and GOT activity to produce OTA and with GPP, to produce CBGA in cells conducive to large scale transgene production and maintenance, and cannabinoid production, such as, in mammal cells, insect cells, yeast cells, bacteria cells, plant cells and so on. Polynucleotides and polypeptides of interest can be obtained from species other than C. sativa.

In embodiments, a host cell is manipulated to comprise three sets of functions, a first set comprises a function to enhance GPP production; a second set comprises a function to form OTA; and a third set comprises a function to form CBGA. Each set can comprise one or more nucleic acids encoding one or more polypeptides with one or more enzymic activities. Those one or more nucleic acids can be carried by one or more vectors. At least one polypeptide is from a species or organism other than a Cannabis. Hence, for example, a first set can comprise a GPPS, a modified FPPS, a GPPS-SSU and so on or combination thereof.

Because GPP is a more ubiquitous and a more conserved metabolite amongst organisms than is OTA, choice of host cell can be flexible as to ensuring enhanced GPP levels in a cell. In embodiments, a host cell may require fewer introductions of foreign sequences (that is, fewer transformation procedures) to obtain or to enhance GPP production.

Similarly, the third set may require a single transformation procedure with a vector comprising a coding sequence that comprises a GOT activity.

In embodiments, the first set comprises a modified FPPS or CS that produces GPP.

In embodiments, the first set comprises a polypeptide with GPPS activity for producing GPP.

In embodiments, the first set comprises a polypeptide that comprises GPPS-SSU activity, along with a separate polypeptide with GPPS-LSU activity, with GGPPS activity, with CS activity or with FPPS activity and so on, to form GPP.

Suitable GGPPS sequences can be obtained from GenBank but also are described in, for example, Kuntz et al. (The Plant J 2(1)25-34, 1992), Wang & Dixon, PNAS 106(24)9914-9919, 2009), Jiang et al. (JBC 270(37)21793-21799, 1995), and Ye et al. (Mol Biol Cell 18:3568-3581, 2007).

Suitable GPPS sequences can be obtained from GenBank but also are described in, for example, Burke & Croteau (JBC 277(5)3141-3149, 2002), Bouvier et al. (The Plant J 24(2)241-252, 2000), Chang et al. (The Plant Cell 22:454-467, 2010), Kampranis & Makris (Comp Struct Biotech J 3:4, 2012) and Rai et al. (Mol Plant 6(5)1531-1549, 2013).

Suitable GPPS-SSU sequences can be obtained from GenBank but also are described in, for example, Burke & Croteau, supra, Chang et al., supra, Orlova et al. (The Plant Cell 21:4002-4017, 2009), Schmidt et al. (Plant Physiol 152:639-655, 2010), Gilg et al. in pine bark beetle (PNAS 102(28)9760-9765, 2005) and Tholl et al. (The Plant Cell 16:977-992, 2004).

Suitable GPPS-LSU sequences can be obtained from GenBank but also are described in, for example, Tholl et al., supra; Chang et al., supra; and Rai et al., supra.

CS is one of the better studied of enzymes and CS molecules of a variety of species have been isolated, studied and cloned, with many of those sequences deposited in GenBank and described in the literature.

Suitable FPPS sequences can be obtained from GenBank but also are described in, for example, Feron et al. (FEBS 271(1,2)236-238, 1990), Fischer et al. (Biotech Bioeng 108(8)1883-1892, 2011) and Pardo et al. (Microb Cell Fact 14:136-144, 2015).

Suitable OAS, OS, OAC and GOT polypeptides can be based on, for example, molecules with homology to C. sativa OAS, OS, OAC and GOT sequences (Marks et al., J Exp Bot 60(13)3715-3726, 2009 and Raharjo et al., Plant Physiol Biochem 42:291-297, 2004; Taura et al., FEBS Lett 583:2061-2066, 2009; Gagne et al., PNAS 109(36)12811-12816, 2012; Flores-Sanchez & Verpoorte, Plant Cell Physiol 49(12) 1767-1782, 2008; and Fellermeier & Zenk, FEBS Lett 427:283-285, 1998, Li et al., Plant Physiol 167:650-659, 2015 and U.S. Pat. Nos. 8,618,355 and 8,884,100). Some of the sequences associated with those publications are deposited in GenBank.

In embodiments, a host cell is transformed to carry a third set function or ability for expressing a polypeptide with GOT activity.

In embodiments, a host cell comprising GOT activity is transformed to carry a second set function or ability for expressing at least one polypeptide for producing OTA. In embodiments, that can occur, for example, by introducing a nucleic acid encoding an OAS that produces OTA. In embodiments, that can occur, for example, by introducing at least two polypeptides, one with OS or TS activity and the other with OAC activity for making OTA.

A cell comprising a second set and a third set and produces polypeptides that comprise GOT activity and produces OTA is termed herein, “a primed cell.”

In embodiments, a primed cell can be transformed with a foreign GPPS-SSU and separately, a foreign GPPS-LSU. The SSU and the LSU can be from the same or different organisms. For example, the SSU can be from a Mentha species, such as, Mentha piperita, Anthirinum maius, C. breweri, a hop, a tobacco, Chinese sage, Catharanthus roseus, a pine bark beetle and so on.

In embodiments, a primed cell can be transformed with a foreign GPPS-SSU.

In embodiments, a primed cell expressing a GPPS-SSU further is transformed with a sequence encoding a foreign GGPPS, an FPPS, a CS and so on.

In embodiments, a primed cell can be transformed with a sequence of a monomer of a foreign homodimeric GPPS, such as, that of Arabidopsis, A. grandis, Picea abies, a species of Lycopersicon, a species of Quercus, C. roseus, a species of Citrus, such as, C. sinensis and so on.

Normally, intracellular compartments can dictate how a compound is used by a cell, for example, a compound may be found in a plastid for producing a pigment or may be routed to the cytoplasm for sterol production.

In embodiments, foreign expressed genes are targeted for expression and function in particular sites of a cell, for example, based on location of compounds that are substrate for an enzyme, location of other compounds that are one of plural substrates of an enzyme, location of other enzymes of a pathway and so on. Such targeted siting or localization, for example, enhances local concentration of reagents and facilitates enzymic and catalytic activity. For example, local concentrations of GPP, OTA and GOT enhance cannabinoid productions. Location of production also may be governed, for example, if one or more products have a negative consequence on the host cell. Targeting of a polypeptide to a particular organelle can comprise use of a targeting peptide specific for an organelle where any reagents or products with adverse impact on a host cells are sequestered and such localization increases effective concentration of reagents.

Plants closely related to Cannabis, such as, hop and rubber provide orthologous enzymes which can be modified to produce cannabinoids. Active site amino acids known to impact substrate specificity or catalysis are replaced to obtain altered or modified enzymes that synthesize cannabinoids or cannabinoid precursors. Orthologous enzymes can be modified, such as, truncated, to alter substrate specificity to produce polypeptides that synthesize cannabinoids.

Homology paradigms and software are used to identify enzymes with selected degrees of homology to a Cannabis enzyme of interest. Portions of a Cannabis enzyme that relate to synthesizing cannabinoids, such as, relating to a reactant binding site or to a catalytic site, are used as probe to inquire of similar sequences of a bank of nucleic acids, such as, GenBank. Identifying the enzyme from which the probe is obtained in the search can serve as a control of search fidelity and/or completeness.

Hence, orthologs of GPPS, GGPPS, FPPS, CS, OAS, OS, OAC and GOT that are found to or are modified to produce cannabinoids are a focus of the subject matter of interest.

For example, hop (H. lupulus) aromatic prenyl transferase (U.S. Pat. No. 8,618,355 or Li et al., supra); C. limon, which comprises a umbelliferone 8-geranyltransferase; a cis PT from rubber (Hevea brasiliensis); Herranis umbratica, related to cacao; Cleome hassleriana, the spiny spider flower; gromwell (Lithspermum erythrorhizon), a herb which contains 4-hydroxybenzoate geranyltransferase; and the like, which, for example, some enzymes already accept geranyl as a substrate, are examples of usable enzymes. For example, in the case of a non-Cannabis geranyl transferase, that enzyme is modified to accept OTA as a substrate, for example, by altering the size of and composition of that portion of the active site of that ortholog that recognizes and binds the ketide.

In embodiments, modification of GPPS, GGPPS, FPPS and CS to alter or to favor the product produced to be GPP can include a single amino acid change in an active site, multiple amino acid changes, domain changes and change of association in quaternary structures, for example, as described herein and in the literature cited.

To enhance production of recombinant polypeptides and to retain recombinant catalytic activity of interest, a full length nucleic acid encoding an enzyme of interest can be modified to obtain, for example, enhanced expression of the nucleic acid, enhanced catalytic activity of the expressed polypeptide and so on. In embodiments, an enzyme of interest can comprise a truncated polypeptide wherein a full length nucleic acid is manipulated to remove non-critical amino acids, for example, those that position the reagents in or at the active site, those that recognize reagents, those that catalyze reactions between reagents and so on. Removable residues can reside at the amino terminus or portion of the full length enzyme. Various strategies can be used to generate truncated sequences. For example, at the nucleic acid level, digestion with an advantageously sited restriction enzyme can remove selected nucleic acid sequences that relate to particular portions of the expressed polypeptide or a nuclease can remove consecutive bases from a terminus; exons can be PCR amplified and ligated to recreate a synthetic gene of interest; bacteriophage μ provides a strategy (Poussu et al., NAR 33(12)e104, 2005) for producing truncated clones; and so on.

In embodiments, codons of a nucleic acid of interest can be altered to contain those favored by a host cell, without altering the encoded amino acid, arising from known preferred codons of a host cell and/or the known degeneracy of the genetic code.

For example, modified nucleic acids encoding a truncated polypeptide comprising an enzymic activity of interest are cloned into a vector that is operable in a host cell of interest, one which is scalable, such as, P. pastoris, and the cells are grown at density in industrial sized reactors for production of commercially viable scale of polypeptides with enzymic activity of interest and hence, of cannabinoids.

Thus, for example, a modified enzyme-encoding sequence can be placed in a yeast vector, such as, a S. cerevisiae vector, such as, pESC-Leu or pESC-URA (addgene, Cambridge, Mass.); or other available commercially yeast vectors, and the recombinant vector is transformed into and maintained in a suitable yeast strain, such as, of S. cerevisiae, using known materials and methods. Transformants are tested for presence of, expression of and function of recombinant enzymic activity, as known in the art, and thus expressing a polynucleotide that produces a polypeptide comprising a desired enzymic activity, are propagated, are amplified and samples are stored. S. cerevisiae can be useful as that organism expresses a GGPPS encoded by the BTS1 locus and an FPPS encoded by the EGR20 locus, and it has been shown that a single amino acid change in FPPS can alter enzyme activity from making FPP to making GPP.

To have cannabinoid production in a host cell other than one of C. sativa, in part, a GOT expression or integration cassette (or vector); and, an OAS expression or integration cassette (or vector); or an OS expression or integration cassette and an OAC expression or integration cassette; or an OAC integration or expression cassette; are placed into the same host cell so that a part of the cannabinoid metabolic pathway is established in the transformed host cell. Normally, cells of many species, plant, animal or microbe, should contain hexanoyl-CoA, malonyl-CoA, 4-coumaroyl-CoA, DMAPP and IPP as endogenous compounds for one or more metabolic pathways.

In embodiments, manipulation of, for example, hexanoyl-CoA, malonyl-CoA, GPP, OTA, OL, DMAPP and IPP pools can increase production of cannabinoids. Hence, for example, hexanoyl-CoA can be added to the main or production reactor or fermenter; malonyl-CoA can be added to the main or production reactor or fermenter; OL can be added to the main or production reactor or fermenter; GPP can be added to the main or production reactor or fermenter; OTA can be added to the main or production reactor or fermenter; DMAPP can be added to the main or production reactor or fermenter; IPP can be added to the main or production reactor or fermenter; and so on; or any combination thereof may be added to the production culture, such as, a continuous culture, to enhance cannabinoid production.

Because some of the involved materials and pathways of interest may be integral to survival of a host cell, additional manipulations ancillary to the particular genes and enzymes disclosed herein may facilitate host cell viability, cannabinoid expression level and so on, see, for example, Ignea et al. (ACS Synth Biol 3(5)298-306, 2014); Brown et al. (PNAS 112(11)3205-3210, 2015); and Ignea et al. Microb Cell Fact 10:4, 2011.)

For example, in embodiments, a gene editing technique can be used to manipulate a coding sequence encoding an activity not directly related to cannabinoid synthesis, such as, not one making GPP or not one making OTA, but for example, one making a component or enzyme for using, for example, GPP for making a terpenoid other than a cannabinoid, or a component or enzyme for making a compound or enzyme for making a GPP for making a terpenoid other than a cannabinoid; one for making a product of a terpenoid or a polyketide pathway; one that would minimize or remove a control or a negative feedback of terpenoid or polyketide synthesis; and so on.

Methods for scaled growth of S. cerevisiae cells are known providing the opportunity of large scale production of cannabinoids at commercial levels.

In embodiments, cannabinoid production can occur in a cell-free environment as the reagents, for example, malonyl-CoA, IPP, DMAPP, 4-coumaroul-CoA and hexanoyl-CoA, are available commercially, OTA and GPP can be made or purchased, OTA can be made with the OAS of interest, OAC of interest or an OS and OAC of interest; GPP can be made with a GPPS of interest, a GPPS-SSU or a modified FPPS or CS and so on, of interest; and a GOT of interest is available. Hence, GOT can be combined with GPP and OTA in a reaction medium and the CBGA produced is removed. That reaction mixture can be configured also to produce OTA and/or GPP; and/or to convert CBGA to a downstream cannabinoid. In embodiments, OTA and/or GPP can be made by enzymatic means in one or separate reactions. In embodiments, converting CBGA to downstream cannabinoids occurs in one or more separate reactions.

Cannabinoids produced as described herein can be used for therapeutic purposes. An effective amount of an active substance and a dosing schedule for a given indication will be readily ascertainable by those skilled in the art through animal modeling and clinical trials; or will track parameters of an already approved drug for a particular indication.

Formulations and devices for drug delivery are known to those skilled in the art. Compositions of the design can comprise an aqueous solution, an inhaled preparation, an oil-based solution, a depot, a cream, an ointment and so on. Said drug delivery depot can comprise a solid matrix from which an active ingredient slowly disperses or a solid matrix that slowly dissolves or degrades thereby releasing an active ingredient therein. Said drug delivery depot can comprise an in situ-formed gel, a drug delivery device, a patch, a contact lens and so on. Compositions can be administered by any means, such as, intravenous, subcutaneous, sublingual, intramuscular and so on, orally, such as, by ingestion of a solid or a liquid, by inhalation, intranasally and so on.

In embodiments, an emulsion-based formulation as disclosed in U.S. Pat. Nos. 5,578,586; 5,371,108; 5,294,607; 5,278,151; and 4,914,088 can be used. Generally, an admixture of a charged phospholipid and a non-polar oil are mixed to form a finely divided oil-in-water emulsion. Another approach is described in U.S. Pat. Nos. 4,818,537 and 4,804,539, where liposome compositions in the form of emulsions are provided.

Hydrophobicity of a compound of interest can be controlled by, for example, chemically modifying a compound, conjugating with a more hydrophilic carrier compound, containing a compound within a formed structure, such as, a liposome, combining a compound with a surfactant or amphiphilic compound, and so on.

What constitutes an effective amount for treatment and/or prophylaxis depends on, among other factors: a particular compound or compounds being administered; residence time provided by a particular formulation of an active substance; species, age and body weight of a subject; particular condition for which treatment or prophylaxis is sought; and/or severity of a condition, for example.

Hence, as known in the art, a compositions of the instant invention can comprise a wide range of components as known in the art. Reference also can be made to U.S. Pat. Nos. 6,013,271; 6,267,985; 4,992,478; 5,645,854; 5,811,111; and 5,851,543. Examples of functional classes of ingredients are antifoaming agents, antimicrobial agents, antioxidants, binders, biological additives, bulking agents, chemical additives, colorants, denaturants, dispersants, deodorants, lubricants, analgesics, fragrances, humectants, thickeners, opacifying agents, plasticizers, preservatives, such as, dichlorobenzyl alcohol, benzoic acid, methylparaben and phenyl, propellants, reducing agents, suspending agents (nonsurfactant), gelling agents, such as, agar, petrolatum and mineral wax, ultraviolet light absorbers, viscosity increasing agents (aqueous and non-aqueous) and so on.

A solution of the design can contain various pharmaceutically acceptable additives, carriers, excipients or diluents as appropriate, such as, buffer, electrolyte, isotonicity agent, preservative, solubilizing agent, stabilizer, chelating agent, thickener, pH adjusting agent and the like.

Aqueous preparations can be made as known in the art; that is, hydrophilic compounds can be dissolved in a water or an aqueous buffer and the preparation sterilized, for example, by filtration, and then aliquoted. Lipoidal formulations can be obtained by dissolving a cannabinoid in a suitable hydrophobic vehicle, such as, an edible oil, an oil suitable for topical use, an organic liquid and so on.

As a buffer, for example, boric acid or a salt thereof (sodium borate etc.), citric acid or a salt thereof (sodium citrate etc.), tartaric acid or a salt thereof (sodium tartrate etc.), gluconic acid or a salt thereof (sodium gluconate etc.), acetic acid or a salt thereof (sodium acetate etc.), lactic acid or a salt thereof (sodium lactate etc.), phosphoric acid or a salt thereof (sodium or potassium hydrogen phosphate, sodium or potassium dihydrogen phosphate etc.), various amino acids, such as, glutamic acid, histidine, lysine, arginine, ε-aminocaproic acid and the like, tris buffer, etc., can be mentioned. Buffers can be used in a combination of one or more kinds thereof.

As electrolytes, for example, sodium chloride, potassium chloride, calcium chloride, magnesium chloride, sodium bicarbonate and the like can be used.

As an isotonicity agent, for example, sorbitol, glucose, mannitol, glycerol, propylene glycol, glycerin, sodium chloride, potassium chloride and the like can be mentioned.

As a preservative, for example, paraoxybenzoates, benzalkonium chloride, thimerosal, phenethyl alcohol, methyl paraben, propyl paraben, benzethonium chloride, benzyl alcohol, sorbic acid or a salt thereof, chlorhexidine or a salt thereof, sodium dehydroacetate, cetylpyridinium chloride, alkyldiaminoethylglycine hydrochloride, chlorobutanol and the like can be mentioned. A combination of one or more kinds or types thereof can be used.

As a solubilizing agent, for example, nonionic surfactants, such as, sorbitan polyoxyethylene fatty acid esters (polysorbate 80 and the like), polyoxyethylene hydrogenated castor oil, polyoxyethylene monostearate (polyoxyl stearate 40 and the like) and like, water-soluble polymers, such as, polyethylene glycol (macrogol 4000 and the like), poloxamers and the like, polyvinylpyrrolidone and the like, propylene glycol, cyclodextrins and the like can be mentioned.

As a stabilizer, for example, disodium edetate, thiosodium sulfate, ascorbic acid, proline, a cyclodextrin, phosphoric acid or a salt thereof, sulfite, citric acid or a salt thereof, dibutylhydroxytoluene and the like can be mentioned. A stabilizer can include, for example, a conventionally known one, such as, hydroxypropylmethylcellulose, polyvinyl alcohol, carboxymethylcellulose, hydroxymethylcellulose and glycerin. A stabilizer can include, for example, an oxidant. A stabilizer also can include, for example, a protein, such as, human serum albumin or gelatin.

A solubilizer includes, for example, polyoxyethylene glycol ethers (e.g. sodium carboxymethylcellulose, polyoxyethylene lauryl ether, polyoxyethylene oleyl ether etc.), polyethylene glycol higher fatty acid esters (e.g. polyethylene glycol monolaurate, polyethylene glycol monooleate etc.), polyoxyethylene fatty acid esters (e.g. polyoxyethylene sorbitan monolaurate, polyoxyethylene sorbitan monooleate etc.) and so on.

An antioxidant includes, for example, ascorbic acid, sodium bisulfite, sodium thiosulfate, thiolated compounds, such as, dithiothreitol, mercaptoethanol, acetylcysteine, glutathione and the like; and so on.

An antiseptic includes, for example, chlorobutanol, benzalkonium chloride, cetylpyridinium chloride and so on.

As a chelating agent, for example, sodium edetate, sodium citrate, phosphoric acid or a salt thereof (sodium phosphate etc.) and the like can be mentioned.

As a thickener, for example, methylcellulose, hydroxyethylcellulose, hydroxypropylcellulose, hydroxypropylmethylcellulose, sodium carboxymethylcellulose, polyvinylpyrrolidone, polyvinyl alcohol, polyethylene glycol, hyaluronic acid and the like can be mentioned.

As a pH adjusting agent, for example, sodium hydroxide, potassium hydroxide, sodium carbonate, sodium hydrogencarbonate, boric acid or a salt thereof (sodium borate), hydrochloric acid, citric acid or a salt thereof (sodium citrate, sodium dihydrogen citrate etc.), phosphoric acid or a salt thereof (sodium dihydrogen phosphate, potassium dihydrogen phosphate etc.), acetic acid or a salt thereof (sodium acetate, ammonium acetate etc.), tartaric acid or a salt thereof (sodium tartrate etc.) and the like can be mentioned.

A formulation of the design can be prepared to have a pH from about 3 to about 10, from about 4 to about 8, from about 5 to about 7 and so on.

A composition of the design may contain or used in conjunction with, insofar as the objects of the invention are not impaired, one or more additional pharmaceutical or active agents to complement or to add to a medicinal impact of a composition of interest, for example, an analgesic, a radiotherapy, a chemotherapy, a neural system (central or peripheral) therapy and the like can be used with a cannabinoid in a combination therapy.

Ointments generally are substantially lipophilic and creams generally comprise an emulsion of both lipophilic and hydrophilic ingredients. An artisan would well recognize how to formulate such preparations to include an active agent as described herein.

Lipids for making an ointment, an oil or a cream can comprise an oil derived from animals, plants, nuts, petroleum etc. Those derived from animals, plant seeds, and nuts are similar to fats and consequently can contain one or a number of one or more polar acids and/or ester groups. Alternatively, oils derived from petroleum are usually aliphatic or aromatic hydrocarbons that are essentially free of polar groups. Other oil-based products that can be used include hydrocarbons or mineral fats obtained by the distillation of petroleum (petroleum jelly); vegetable oils and liquid triglycerides; animal fats or solid natural triglycerides; and waxes or solid ethers of fatty acids and organic alcohols. Lanolin or wool fats that are obtained from sheep wool and made up of fatty acids and cholesterol esters; and cetyl and stearyl alcohols, which are solid alcohols obtained by hydrogenation of respective acids are also useable. Amphiphilic compounds, such as, soaps or salts of fatty acids, that may be acidic or basic depending on whether the hydrophilic group is anionic or cationic, sulfated alcohols which can be semi-synthetic substances and synthetic surface active agents are known in the art and can be used in a preparation of interest for the intended use, for example, as a dispersing agent. Glycerin has hydrophilic properties and hence, is useful as a humectant in a preparation of interest.

Materials that may be used in a topical preparation of interest include liquid alcohols, liquid glycols, liquid polyalkylene glycols, liquid esters, liquid amides, liquid protein hydrolysates, liquid alkylated protein hydrolysates, liquid lanolin, lanolin derivatives and other like materials. Particular examples include monohydric and polyhydric alcohols, e.g., ethanol, isopropanol, glycerol, sorbitol, mannitol, cetyl alcohol and propylene glycol; ethers; polyethylene glycols; methoxypolyoxyethylenes; carbowaxes having molecular weights ranging from 200 to 20,000; polyoxyethylene glycerols; polyoxyethylene; sorbitols; stearoyl diacetin and so on.

A number of different emulsifiers or surfactants can be used to prepare an ointment or a cream of interest. Emulsifiers can be ionic or non-ionic. Examples of amphoteric surfactants and anionic surfactants useful in the compositions of the design include those disclosed in McCutcheon's, “Detergents and Emulsifiers”, North American edition (1986) and McCutcheon's, “Functional Materials”, North American Edition (1992); both of which are incorporated by reference herein in entirety. Surfactants that can be used include a betaine, a sultaine and a hydroxysultaine. Examples of other amphoteric surfactants are alkyliminoacetates, iminodialkanoates and aminoalkanoates.

A lipoidal preparation, such as, an oil, can be comprised substantially of an oil which is pharmaceutically acceptable. That lipoidal vehicle can comprise a petrolatum or a paraffin. A mineral oil can be used to attain a flowable consistency, for example. Also, water, lanolin and so on can be included, see U.S. Pat. No. 5,470,881, for example.

A cream comprises an emulsion of lipophilic and hydrophilic components, which are mixed into stable forms. Hence, lipophilic components are mixed to form a solution, and hydrophilic components are mixed to form a solution. Then the two solutions are mixed, one into the other as a design choice, to obtain a stable emulsion. Generally, creams containing more water can be thinner and may disperse more readily at warmer temperatures.

The compositions can take the form of tablets, pills, capsules, powders, sustained-release formulations, depots and the like. The composition can be formulated as a suppository, with traditional binders and carriers, such as, triglycerides. Oral formulations can be liquid or solid, and can include standard carriers, such as, pharmaceutical grades of mannitol, lactose, starch, magnesium stearate, a flavorant, sodium saccharine, cellulose, magnesium carbonate etc.

Examples of suitable carriers are described in, for example, “Remington's Pharmaceutical Sciences,” Martin. Such compositions will contain an effective amount of a cannabinoid or a combination of cannabinoids together with a suitable amount of carrier so as to provide a form for proper administration to a patient. As known in the art, a formulation will be constructed to suit a mode of administration.

Systemic administration also can be by transmucosal or transdermal means. For transmucosal or transdermal administration, penetrants appropriate to the barrier to be permeated are used in a formulation. Such penetrants generally are known in the art and include, for example, for transmucosal administration, detergents, bile salts and fusidic acid derivatives and so on. Transmucosal administration can be accomplished through use of nasal sprays or suppositories. For transdermal administration, active compounds can be formulated into ointments, salves, gels or creams as generally known in the art. A suitable carrier includes dimethylsulfoxide. A formulation can be applied to a flexible and adhesive carrier for application to skin in the form of a patch where active ingredient included in or associated with an adhesive is placed in direct contact with skin to enable penetration of active ingredient across the skin.

Compositions of the design may be stored as known in the art, for example, oils and emulsions can be stored at room temperature, liquid hydrophilic preparations can be refrigerated or frozen, may be lyophilized and so on.

A composition of interest is administered to a patient in need of treatment, for example, one with a neurologic disorder, one suffering from nausea and so on, at a desired dosing and route of administration as determined empirically and as known in the art.

The invention now will be exemplified in the following non-limiting examples.

### EXAMPLES

### Example 1

Standard recombinant DNA and molecular cloning techniques used herein are known in the art and are described, for example, in Sambrook, et al. (supra); Silhavy et al., “Experiments with Gene Fusions,” Cold Spring Harbor Laboratory Press, Cold Spring, N.Y. (1984); and Ausubel et al., “Current Protocols in Molecular Biology,” Greene Publishing Assoc. and Wiley-Interscience (1987).

Nucleotide and amino acid percent identity and similarity comparisons can be made using the GCG suite of programs, applying default parameters, unless indicated otherwise.

### Example 2

C. sativa plants are grown under hydroponic conditions in a growth chamber. Approximately 5 g of mature female inflorescences 8 weeks after onset of flowering are collected. Tissue is macerated in phosphate-buffered saline (PBS) using a blender. Material is sieved and the flow-through is centrifuged. After another wash, pellets are resuspended in 100 μl of PBS. A suspension is estimated to contain about 100,000 intact glands.

Total RNA is isolated from the glands and about 100 ng of total RNA are used to make a cDNA library using a commercially available kit. Vectors can be configured to allow directional cloning of cDNA inserts. Plasmid DNA is isolated from selected clones and the inserts sequenced, for example, using an M13 forward primer that reads into 5′ end of the inserts.

Full length cDNA's with insert sequences substantially or identical to the TS sequence of Taura et at, supra, are isolated.

Polyketide synthase (PKS) assay for forming OL (160 μl reaction volume) contains 40 μl of enzyme, 100 mM sodium citrate buffer (pH 5.5), 200 μM malonyl-CoA and 100 μM hexanoyl-CoA. Boiled polypeptide as negative control is assayed in parallel with all reactions. All negative controls show a lack of OL formation. Reactions are incubated for 0.5 h at 30° C., 200 μl methanol are added to the reaction and a 100 μl aliquot is use to identify products by HPLC.

Clones producing TK or OL are selected as OS or TS clones.

### Example 3

Full length cDNA's with insert sequences substantially or identical to GenBank accession JN 679224 are isolated.

OAC assay for forming OTA (50 μl reaction volume) contains 40 μl of enzyme, 20 mM Hepes buffer (pH 7), 5 μM DTT, 0.2 mM hexanoyl-CoA, 12 μg malonyl-CoA synthase, 0.2 mM CoA, 0.4 mM ATP, 2.5 mM MgCL₂, 8 mM sodium malonate, TS and candidate OAC polypeptide. Boiled polypeptide as negative control is assayed in parallel with all reactions. All negative controls show a lack of OAC formation. Reactions are incubated for 1 h at 20° C. Products are identified by HPLC and/or MS.

Clones producing OTA are selected as OAC clones.

### Example 4

The gland library of Example 2 is used to recover prenyl transferase sequences.

The library is screened using probes carrying sequences of consensus or conserved portions of similar prenyl transferases, such as, that of hop aromatic prenyl transferase (U.S. Pat. No. 8,618,355) or of the sequence provided in U.S. Pat. No. 8,884,100, and those with homology with the probes are isolated.

Yeast microsome preparations are obtained as known in the art. The standard assay reaction contains 1 mM dithiothreitol, 10 mM Tris-HCl (pH 7), 10 mM MgCl₂, 30 mM NaF as a phosphatase inhibitor, 0.2 mM OTA, 1 mM GPP and 5 μL of microsomes. Reaction mixtures are incubated in a total volume of 250 μL at 30° C. and are extracted twice with 250 μL of ethyl acetate. Organic phases of the extracts are combined and dried under nitrogen gas. Extracts then are dissolved in 250 μL of 40% (w/v) methanol and 1 μL of the resulting solution is analyzed with HPLC TOF MS to identify presence of any reaction products and identity of any produced products.

Clones producing CBGA are selected as GOT clones.

### Example 5

A GOT clone (of Example 4, or by any other method) is selected. A restriction site located in the first 387 bases at the 5′ end of the coding sequence and nowhere else in the coding sequence is used for controlled cleavage of and removal of 5′ terminal sequences of the GOT coding sequence to form a truncated core GOT nucleic acid.

The truncated nucleic acid is cloned into a vector following reconstruction of a 5′ cloning end, the vector is placed in a cell, propagated, the insert sequenced and expressed. The expressed truncated GOT is tested for ability to produce CBGA as provided in Example 3. The level of transferase activity is ascertained.

Clones producing CBGA at a level at least 80% of wild type GOT as, for example, observed in the assays of Example 3 are identified as truncated core GOT clones.

### Example 6

A GOT clone of Example 4 is selected. A restriction site located between bases 388 and 522 of the coding sequence and nowhere else in the coding sequence is used for controlled cleavage of and removal of 5′ terminal sequences of the GOT coding sequence to form a truncated active site GOT nucleic acid.

The truncated nucleic acid is cloned into a vector following reconstruction of a 5′ cloning end, the vector is placed in a cell, propagated, the insert sequenced and expressed. The expressed truncated GOT is tested for ability to produce CBGA as provided in Example 3. The level of transferase activity is ascertained.

Clones producing CBGA at a level at least 80% of wild type GOT as, for example, observed in the assays of Example 3 are identified as truncated active site GOT clones.

### Example 7

To stabilize expression of a GOT nucleic acid, a GOT vector is integrated in the genome of S. cerevisiae by homologous recombination, DeMarini et al. (supra). Integration sites are, for example, homologous to ura3 locus sequences, pDC6 sequences (encoding an isozyme of pyruvate decarboxylase of minor activity) or sequences of a non-coding region, and nucleic acids fragments hybridizable therewith are ligated to the 5′ and 3′ ends of the vector comprising a GOT sequence. The construct is inserted into S. cerevisiae known to have sequences homologous to those added to the GOT vector. Transformants are selected, cultivated in a suitable medium and genomic nucleic acids are isolated to determine whether the GOT sequence is integrated into the yeast genome. Restriction mapping can confirm the exact location of the GOT sequence.

A strain with GOT sequences included in the host genome is amplified and then tested for cannabinoid production. The strain then is tested for growth in larger scale fermenters.

### Example 8

For efficient accumulation of hydrophobic cannabinoids, continuous or multi-phase fermentation and a biocompatible organic solvent is used. Baffled, cotton stopped, 500 ml Erlenmeyer flasks with 100 ml of defined minimal medium and 15 g/L galactose are inoculated with an overnight culture of a GOT expressing strain, made by transforming a yeast strain of Example 5 with vectors as obtained in Examples 2 and 3, and the strain is maintained under rotation culture. When OD at 600 nm of 1 is reached, 10 ml of dodecane (equivalent to 10% of the culture medium) are added to the shake flask. Dodecane does not have a negative impact on cell growth. At the end of logarithmic phase, once galactose is depleted, a sample is taken, medium of the sample is centrifuged and the upper organic phase is analyzed by HPLC or MS for cannabinoids. The cell pellet is lysed with detergent and the lysate is analyzed by HPLC or MS for cannabinoids.

Cannabinoids are found in the medium and in the cell pellet.

The remainder of the culture is added to a production fermenter and maintained under rotation culture. Dodecane at the same rate is added to the production fermenter.

### Example 9

For expression in insect cells, the truncated core GOT vector of Example 5 is placed into an sf9 vector using, for example, the InsectDirect system and GeneJuice of MilliporeSigma (Burlington, Mass.). Following culture, presence of GOT sequences in sf9 cells is checked by hybridization. Sf9 cells containing GOT sequences are then transformed with an sf9 vector carrying the OAC sequence of Example 3, prepared in the same manner as for the GOT construct. Following culture, cells carrying both GOT and OAS sequences are selected for further growth and expansion. Cells are tested for expression of GOT and OAS polypeptides, and those cells expressing both polypeptides are lysed and tested for cannabinoid presence by HPLC and MS.

CBGA is found in the insect cell lysate.

### Example 10

A full length cDNA which is substantially or is identical to the Abies grandis GPPS, AgGPPS2 of Burke & Croteau (Arch Biochem Biophys 405(1)130-136, 2002) is placed into an S. cerevisiae background, such as, strain BY4741. Yeast are made competent and are transformed with the vector of interest practicing known materials and methods. Yeast are incubated in, for example, YPD or a synthetic complete drop-out (SC) medium with 2% (wt/vol) glucose lacking specific amino acids (available, for example, from Clontech/Takara, Sigma-Aldrich and so on) and incubated at 30° C. Transformation success is assessed by selection and hybridization with suitable probes.

Expression of GPPS activity is determined by production of GPP using thin layer chromatography (TLC) to separate prenol products, as known in the art, and as taught in Bouvier et al., supra. Briefly, in a volume of 0.5 ml containing 5 μM [¹⁴C]IPP (isopentyl pyrophosphate), 10 mM MgCl₂, 0.2 mM MnCl₂, 2 mM DTT and 10 μM DMAPP, GPP, FPP or GGPP are added 250 μl of putative enzyme solution. Following incubation for 1 hour at 30° C., the reaction products are dephosphorylated and analyzed, using unlabeled authentic prenol standards.

GPP is detected.

### Example 11

AgGPPS2 of Example 10 is codon optimized for residence and expression in S. cerevisiae (see, for example, GeneScript, Piscataway, N.J.), using, for example, site directed mutagenesis to replace bases, for example, using modified PCR primers, PCR primer extension and so on, as known in the art.

The codon optimized sequence is cloned and introduced into an S. cerevisiae host, which is propagated and then is assessed for expression of GPPS, for example, by nucleic acid hybridization, for example, a Northern blot to determine message levels, by SDS-PAGE to assess GPPS levels or by GPP production to determine levels of enzyme activity.

GPP is detected.

### Example 12

Full length cDNA's with insert sequences substantially or identical to Ips pini GPPS are isolated, Gilg et al., supra.

The coding sequence is cloned into a suitable yeast expression vector which is transformed into a suitable yeast host. The transformed yeast is propagated and levels of GPPS coding sequence, GPPS message, GPPS levels and amounts of GPP produced are assessed as described in Examples 10 and 11.

GPP is detected.

### Example 13

To a yeast cell expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the AgGPPS2 coding sequence of Example 11 using a suitable yeast vector. That transformed primed yeast cell is tested for AgGPPS2 presence, expression and activity as provided in Example 10.

The transformed primed cell is incubated under standard conditions and tested for cannabinoid production.

CBGA is detected.

### Example 14

A Bacillus stearothermophilus farnesyl diphosphate synthase (FPPS) (Koyama et al., J Biochem 113:355-363, 1993) carrying a substitution of serine 82 with phenylalanine is cloned into a suitable E. coli vector and then placed into a suitable E. coli host, such as, DH5a. The cells are propagated and levels of enzyme activity are obtained as provided in Narita et al., J Biochem 126:566-571, 1999.

Prenyltransferase activity is determined essentially as provided in Example 10, and as provided in Narita et al.

GPP is detected in the cell lysate.

### Example 15

A PaIDS1 coding sequence of Picea abies carrying two amino acid substitutions, M175I and P174C (Schmidt et al., supra), is cloned into a suitable yeast vector and transformed into a suitable yeast host. The cells are propagated, located and lysed.

GPP is detected in the cell lysate.

### Example 16

The ERG20 locus of S. cerevisiae encodes an FPPS. It is known S. cerevisiae does not have a GPPS and that the FPPS can make GPP but does not release the GPP which remains bound at the enzyme catalytic site, likely serving as an intermediate to be converted into FPP (Fischer et al., supra).

Plasmid pLB41, which carries the S. cerevisiae FPPS coding sequence (Blanchard & Karst, Gene 125:185-189, 1993), is exposed to a site directed mutagenesis treatment using a commercially available kit (for example, from ThermoFisher, New England Biolabs and so on) using primers that frame position 197 to introduce an amino acid substation at amino acid 197 changing the Lys at that site in the wild type polypeptide to a Gly.

Yeast strain AE9 (Fischer et al., supra) is transformed with the pLB41 plasmid carrying the K197G mutation and is propagated on minimal medium and selected for Trp and G418 resistance. Selected cells then are propagated on YPD or on a minimal medium with supplementation.

Yeast cells are expanded in shake culture using minimal liquid medium and grown to stationary phase. Cells are opened and terpenols are extracted with n-pentane and exposed to GC-MS analysis.

While the strain grows a little slower than wildtype, the mutant produces high levels of GPP.

### Example 17

The PaIDS2 sequence of P. abies (Schmidt et al., supra) is exposed to site directed mutagenesis as provided in Example 16 to introduce two mutations, F96W and N127W into the bifunctional GPPS and GGPPS polypeptide (Ignea et al., 2014, supra).

Terpenoids are characterized as provided in the previous Examples.

GPP is detected.

### Example 18

A domestic fowl homodimeric FPPS carrying the amino acid substitution N144W (Fernandez et al., supra) is codon optimized for S. cerevisiae as provided in Example 11 and as known in the art. The codon optimized insert is cloned into a high copy number yeast expression vector and is transformed into S. cerevisiae as described herein and as known in the art.

The cells are lysed and the lysate tested for terpenoids as described herein or as known in the art, for example, the soluble portion of the lysate is dried, suspended in MeOH and analyzed by LC-MS, by a dodecane overlay method (Ignea et al., supra) and so on.

FPP production is curtailed or non-existent and GPP is present.

### Example 19

To the primed cell of Example 13 is added the coding sequence of the B. stearothermophilus FPPS of Example 14 using a yeast vector.

That transformed primed yeast cell is tested for B. stearothermophilus FPPS presence, expression and activity as provided in Examples 10 and 14.

The transformed primed yeast cell is incubated under standard conditions and is tested for cannabinoid production.

CBGA is detected.

### Example 20

To the primed cell of Example 13 is added the PaIDS1 coding sequence of Example 15 using a yeast vector.

That transformed primed cell is tested for PaIDS1 presence, expression and activity as provided in Examples 10 and 15.

The transformed primed yeast cell is incubated under standard conditions and is tested for cannabinoid production.

CBGA is detected.

### Example 21

To the primed cell of Example 13 is added plasmid pLB41 of Example 16. The transformed primed yeast cell is cultivated and is tested for cannabinoid production as known in the art or as taught herein.

CBGA is detected.

### Example 22

To the primed cell of Example 13 is added the mutated domestic fowl FPPS coding sequence of Example 18 using a yeast vector. The transformed primed cell is tested for presence of the domestic fowl coding sequence and operability of the mutated domestic fowl FPPS.

The transformed primed yeast cell is cultivated, the cells are collected, are lysed and are tested for cannabinoid production as taught herein or as known in the art.

CBGA is detected.

### Example 23

A full length cDNA which is substantially or is identical to the C. annuum GGPPS (Kuntz et al., supra) is treated to remove the N terminal about 60 amino acid transit peptide. That remaining open reading frame is placed in a suitable high copy number expression vector and is placed into an S. cerevisiae background, such as, strain BY4741. Yeast are made competent and transformed with the vector of interest practicing known materials and methods. Yeast are cultured in YPD or a synthetic complete drop-out (SC) medium with 2% (wt/vol) glucose lacking specific amino acids (available, for example, from Clontech/Takara, Sigma-Aldrich and so on) and incubated at 30° C. Transformation success is assessed by selection and hybridization with suitable probes.

Expression of GGPPS activity is determined by production of products using thin layer chromatography (TLC) to separate prenol products, as known in the art, and as taught in Kuntz et al., supra. Briefly, in a volume of 0.1 ml, 50 mM Tris buffer, containing 0.5 μCi [¹⁴C]IPP, 5 mM MgCl₂, 1 mM MnCl₂, 2 mM DTT and 10 μM of DMAPP, GPP, FPP or GGPP are added 5-200 μg of putative enzyme solution. Following incubation for 0.5 hour at 25° C., the reaction products are dephosphorylated, extracted and samples analyzed in a scintillation counter. The samples also are exposed to TLC and autoradiography using unlabeled authentic prenol standards. Samples also can be analyzed by reversed phase chromatography.

GGPP is detected.

### Example 24

The coding sequence of the spo9 locus of S. pombe (Ye et al., supra) is placed in a high copy number E. coli expression vector and is used to transform a suitable E. coli host. The E. coli host also is transformed with a vector carrying the S. pombe fsp1 open reading frame. The doubly transformed host carries both spo9 and fsp1 sequences.

Cells are grown to log phase in LB medium, collected by centrifugation and ruptured by sonication. The supernatant is tested for prenyl diphosphate synthase activity. The assay reaction mixture contains 1 mM MgCl₂, 0.1% Triton X-100, 50 mM potassium phosphate buffer (pH 7.5), 10 μM C¹⁴-IPP, 5 μM FPP and 200 μg of enzyme extract in a final volume of 0.4 ml. The mixture is incubated for 1 hr at 30° C. Reaction products are extracted with 1-butanol-saturated water and hydrolyzed with acid phosphatase. The mixture is extracted with hexane and is analyzed by reverse phase TLC. Radioactivity is detected with an imaging analyzer.

GGPP is detected.

### Example 25

The spo9 coding sequence of Example 24 is placed into a vector compatible with S. cerevisiae. The vector is used to transform an S. cerevisiae. Presence of spo9 sequences in the transformant is confirmed.

The transformant is treated using a gene editing methodology to inactivate the endogenous Bts1 locus of S. cerevisiae, for example, using Crispr materials and methods, available commercially, for example from Synthego (Redwood City, Calif.), GeneCopeia (Rockville, Md.), Origene (Gaithersburg, Md.) and so on.

The yeast cell carrying spo9 with an inactive Bts1 locus is propagated and tested for prenylated species.

GGPP is detected.

### Example 26

H. lupulus GPPS 30 kD (SSU and 40 kD LSU (Wang & Dixon, supra) each is cloned into a yeast expression vector. The transit peptide targeting the polypeptide to a plastid is removed prior to cloning. A suitable S. cerevisiae is transformed with both vectors. Presence of the vector inserts in the propagated yeast is confirmed by hybridization using suitable probes.

The transformed yeast is grown and then treated as provided in Example 10 and as known in the art to ascertain terpenoid production.

GPP is detected.

### Example 27

H. lupulus GGPPS 30 kD SSU and 37 kD LSU (Wang & Dixon, supra) each is cloned into a yeast expression vector. The chloroplast/plastid transit peptide is removed prior to cloning. S. cerevisiae is transformed with both vectors and presence of insert coding sequences is confirmed with suitable probes.

The transformed yeast is grown and is treated as in Example 26 for terpenoids.

GPP and GGPP are detected, in about a 60:40 distribution.

### Example 28

Peppermint is the source of GPPS SSU and LSU (Burke et al., PNAS 96:13062-13067, 1999). The coding sequence of the 28 kD SSU and of the 37 kD LSU, with the N terminal transit peptide removed, are cloned into S. cerevisiae high copy expression vectors.

Yeast are grown to log phase and then are collected. Cells are lysed by sonication and the lysate is separated by centrifugation to yield a supernatant.

To 10 μl of supernatant are added 70 μl of MOPSO buffer (25 mM, pH 7) containing 10% glycerol, 10 mM MgCl₂ and 1 mM DTT. DMAPP (10 μM) and C¹⁴—IPP (7 μM) to a 100 μl total volume. The mixture is overlaid with 1 ml of pentane and incubated for 1 hr at 31° C. Then, 10 μl of 3 N HCl are added and the mixture is incubated at the same temperature for an additional 20 min. The contents are mixed and are allowed to separate. The pentane is removed and the aqueous remainder is extracted with 2 ml of diethyl ether. The mixture is tested by scintillation counting. The organic extract is dried over anhydrous sodium sulfate, diluted with a solution of internal standards, concentrated and analyzed by radio-GC.

GPP is detected.

### Example 29

The Mentha GPPS SSU coding sequence of Example 28 is placed into an E. coli compatible vector. The T. canadenssis GGPPS coding sequence (Hefner et al., Arch Biochem Biophys 360:62-74, 1998) and the A. grandis GGPPS coding sequence (Tholl et al., Arch Biochem Biophys 386:233-242, 2001) with the N terminal transit peptide removed, each is cloned into an E. coli compatible expression vector.

When DMAPP and IPP are exposed to a cell carrying the T. canadensis GGPPS or the A. grandis GGPPS, GGPP is detected.

The GPPS SSU clone and one of the other vectors is transformed into a receptive E. coli host, which is screened for the relevant selection factors and is grown in LB broth and then is expanded in a 1 liter culture at 20° C. until A₆₀₀ reached 0.6. The temperature is reduced to 15° C. and then the culture is exposed to 0.4 mM IPTG and culture is continued for another 16 hrs.

The cells are lysed and then the supernatant is examined for prenyltransferase activity as described in Example 28.

Coexpression of the Mentha GPPS SSU and the T. canadensis GGPPS in a cell and exposure to DMAPP and IPP yields GPP.

Coexpression of the Mentha GPPS SSU and the A. grandis GGPPS in a cell and exposure to DMAPP and IPP yields GPP.

### Example 30

To a yeast cell expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the GPPS sequence of Arabidopsis thaliana (Bouvier et al., supra) without the transit peptide using a suitable vector. That transformed primed yeast cell is tested for GPPS coding sequence presence (for example, by hybridization using a suitable probe), expression and activity as provided in Example 10.

The transformed primed yeast cell is incubated under standard conditions and tested for cannabinoid production.

CBGA is detected.

### Example 31

The snapdragon (Antirrhinum majus) GPPS SSU coding sequence (Tholl et al., 2004, supra) is placed into an E. coli compatible vector. The M. piperita GPPS LSU coding sequence of Example 28 with the N terminal transit peptide removed is cloned into an E. coli compatible expression vector.

The A. majus GPPS SSU clone and the vector carrying the mint GPPS LSU are transformed into a receptive E. coli host, which is screened for the relevant selection factors, grown in LB broth and then expanded in a 1 liter culture at 20° C. until A₆₀₀ reached 0.6. The temperature is reduced to 15° C. and then the bacterial culture is exposed to 0.4 mM IPTG and culture is continued for another 16 hrs.

The cells are lysed and then the supernatant is examined for prenyltransferase activity as described in Examples 28 and 29.

Coexpression of the snapdragon GPPS SSU and the mint GPPS LSU in a cell and exposed to DMAPP and IPP yields GPP.

### Example 32

The A. majus GPPS SSU coding sequence of Example 31 is cloned into a plant vector, such as, pCAMBIA 1303 vector (Cambia, Canberra, AU) and is placed into an A. tumefaciens strain. The A. tumefaciens is used to generate transgenic tobacco plants by standard procedures. Plants which are rooted on hygromycin selection are screened for vector sequence presence. Transformants are grown, pollinated, seeded and adapted to greenhouse conditions.

Volatile terpenoids are collected from transgenic plants and are analyzed by gas chromatography (GC)-mass spectrometry (MS) (Dudarova et al., PNAS 102:933-938, 2005). Normally, tobacco does not produce substantial levels of volatile terpenoids from leaves or flowers. Omicene levels are 25 times higher in leaves of transgenic plants expressing GPPS SSU. Leaves also produce a new terpene, myrcene. Transgenic flowers produce omicene whereas control flowers do not. Methyl jasmonate exposure greatly increases the amount of omicene emitted by leaves in controls and in transgenics.

Tobacco sequences with homology to A. majus GPPS LSU are mined from publicly available resources. Two candidates, with N terminal plastid targeting sequences removed, are cloned and are expressed in E. coli and produce GGPP, as analyzed as described herein, for example, in Examples 28 and 29, and as known in the art.

Coexpression of the snapdragon GPPS SSU and either of the two tobacco GGPPS candidates in E. coli and exposure to DMAPP and IPP yields GPP.

### Example 33

The hop GPPS SSU coding sequence of Example 26 is placed into an E. coli compatible vector. The human GGPPS coding sequence (GenBank Acc. No. AAH05252) is cloned into an E. coli compatible expression vector.

When DMAPP and IPP are introduced to a cell carrying the human GGPPS, GGPP is detected.

The hop GPPS SSU clone and the human GGPPS clone are introduced into a receptive E. coli host, which is screened for the relevant selection factors, grown in LB broth and then is expanded in a 1 liter culture at 20° C. until A₆₀₀ reached 0.6. The temperature is reduced to 15° C. and then the culture is exposed to 0.4 mM IPTG and culture is continued for another 16 hrs.

The cells are lysed and then the supernatant is examined for prenyltransferase activity as described in Examples 28 and 29.

GPP is detected.

### Example 34

To a yeast cell expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the hop GPPS SSU sequence of Example 26 and the C. annum GGPPS coding sequence of Example 23, each in yeast compatible vectors. The transit peptide is removed and in embodiments, the inserts comprise codons which are optimized for expression in yeast cells. That transformed primed yeast cell is tested for GPPS SSU and GGPPS coding sequence presence (for example, by hybridization using a suitable probe), expression and activity as provided in Examples 10, 23 and 26.

The transformed primed cell is incubated under standard conditions and tested for cannabinoid production.

CBGA is detected.

### Example 35

To a yeast cell expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the mint GPPS SSU sequence of Example 28 and either of the two tobacco GGPPS coding sequence of Example 32, each in yeast compatible vectors. The transit peptide is removed and in embodiments, the inserts comprise codons which are optimized for expression in yeast cells. That transformed primed cell is tested for GPPS SSU and GGPPS coding sequence presence (for example, by hybridization using a suitable probe), expression and activity as provided in Examples 10, 28 and 30.

The transformed primed cell is incubated under standard conditions and tested for cannabinoid production.

CBGA is detected with either tobacco GGPPS sequence.

### Example 36

To a yeast cell with an intact Bst1 locus expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the mint GPPS SSU sequence of Example 28 in a yeast compatible vector. The transit peptide is removed and in embodiments, the insert comprises codons which are optimized for expression in yeast cells. That transformed primed cell is tested for mint GPPS SSU coding sequence presence (for example, by hybridization using a suitable probe), expression and activity as provided in Examples 10 and 28.

The transformed primed cell is incubated under standard conditions and tested for cannabinoid production.

CBGA is detected.

### Example 37

To a yeast cell with an intact Bst1 locus expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the hop GPPS SSU sequence of Example 26 in a yeast compatible vector. The transit peptide is removed and in embodiments, the insert comprises codons which are optimized for expression in yeast cells. That transformed primed yeast cell is tested for hop GPPS SSU coding sequence presence (for example, by hybridization using a suitable probe), expression and activity as provided hi Examples 10 and 26.

The transformed primed cell is incubated under standard conditions and tested for cannabinoid production.

CBGA is detected.

### Example 38

To a yeast cell expressing the TS of Example 2 and the OAC of Example 3 is added the coding sequence of GOT of Example 4 to produce a primed yeast cell.

The primed yeast cell is tested for OTA production as in Example 3.

OTA is produced.

To the primed yeast cell is added the mint GPPS SSU sequence of Example 28 and the hop GPPS LSU coding sequence of Example 26, each in yeast compatible vectors. Any transit peptide is removed and in embodiments, the inserts comprise codons which are optimized for expression in yeast cells. That transformed primed yeast cell is tested for mint GPPS SSU and hop GPPS LSU coding sequence presence (for example, by hybridization using a suitable probe), expression and activity as provided hi Examples 10, 26 and 28.

The transformed primed cell is incubated under standard conditions and tested for cannabinoid production.

CBGA is detected.

While some of the embodiments of the present invention have been described in detail in the above, those of ordinary skill in the art can make various modifications and changes to the particular embodiments shown without substantially departing from the novel teaching and advantages of the present invention. Such modifications and changes are encompassed in the spirit and scope of the present invention as disclosed herein.

All references cited herein are herein incorporated by reference in entirety.

1. A composition comprising:
(a) a first set comprising at least one polypeptide for enhancing geranyl phosphate (GPP) synthesis; and
(b) a second set comprising a polypeptide comprising geranylpyrophosphate:olivetolate geranyltransferase (GOT) activity;
wherein at least one polypeptide of said first set is obtained from an organism other than a Cannabis sativa.

2. The composition of claim 1, wherein said at least one polypeptide of said first set is truncated or comprises an amino acid substitution in an active site of said at least one polypeptide.

3. The composition of claim 1, where said organism comprises a hop, a species of Mentha, a species of Citrus, a snapdragon or a species of Salvia.

4. The composition of claim 1, wherein said first set comprises a geranyl pyrophosphate synthase (GPPS).

5. The composition of claim 1, wherein said first set comprises a GPPS small subunit (SSU).

6. The composition of claim 1, wherein said first set comprises a geranylgeranyl pyrophosphate synthase (GGPPS).

7. The composition of claim 1, wherein said first set comprises a GPPS large subunit (LSU).

8. The composition of claim 1, wherein said first set comprises a modified farnesyl pyrophosphate synthase (FPPS).

9. The composition of claim 1, wherein said TS is from an organism other than a Cannabis.

10. A yeast or an insect cell comprising the composition of claim 1.

11. The yeast cell of claim 10, comprising a Saccharomyces cerevisiae.

12. A method of making a cannabinoid, comprising:
a. culturing the cell of claim 10 in a seed reactor to form a seed culture;
b. culturing a portion of said seed culture in a production reactor to form a production culture; and
c. collecting cannabinoids from said production culture.

13. The method of claim 12, wherein said cell comprises a Saccharomyces cerevisiae.

14. The method of claim 12, wherein said production reactor comprises a continuous culture.

15. The method of claim 12, wherein said production reactor comprises an organic solvent.

16. The method of claim 12, wherein malonyl-CoA, dimethylallyl pyrophosphate, isopentyl pyrophosphate, 4-coumaroyl-CoA, olivetol, OTA, hexanoyl-CoA or a combination thereof are added to said production reactor.

17. The method of claim 12, wherein GPP is added to said production reactor.


SEQUENCE LISTING (11 sequences)

SEQ ID NO: 1 - protein, 366 aa, Antirrhinum majus
  snapdragon geranyl diphosphate synthase
MRSMNLVDSWAAQVCSVFNQTIPNKTFTGFMKIPVKNPSFLKQKRPFSALSVSAIATKEDEKVKEVETQKEQFNFKTYVAEKAISVNKALDEAVIVKDPPVIHEAMRYSLLAGGKRVRPMLCLAACELVGGDQSNAMPAACAVEMIHTMSLIHDDLPCMDNDDLRRGKPTNHKVYGEDVAVLAGDSLLAFAFEYIATATAGVSPSRILAAIGELAKSIGTEGLVAGQVADIACTGNPNVGLDTLEFIHIHKTAALLEASVVLGAILGGGTDEEVEKLRIFARCIGLLFQVVDDILDVTKSSEVLGKTAGKDLAVDKTTYPKLLGLEKAKEFAAELNRDAKQQLVEFDPHKAAPLIALADYIAYREN

SEQ ID NO: 2 - protein, 407 aa, Citrus limon
  Full=Coumarin 8-geranyltransferase Citrus Limon
MLQMHSNSSFSPKCYYPLQHAGCVKTLQLPLTKVHGGLNRSESKNYAIKCTQSDSFYSTNKIRNNENSSSRNCKPFNKYRVAVTLQQQDCASNNEDDINSTSFRDVLLKKLHALYVFTRPFAMIGTIVGITSIAILPLQSFADLTPKYFMEFLKALLSAVLMNNYVGTVNQVADVEIDKVNKPGLPLASGDLSVGTGLAITLILSLTSLAIALSLQSPPLIFGLIVWFLLGTAYSVDLPFLRWKTNPFLAGMCMVIVFGLVYQFSFFIHFQKYVLGRPVVITRPLIFAAAIISTISAVMSLLKDIPDEDGDKQFGYQSISSKLGKENVLRLCVYALFFAYGVAVIVGASSSFQLGKLVSIIGHSTLAFLLWLRAQTVDLSNNASTYSFYMFIWKLFYAEYLLIHFLR

SEQ ID NO: 3 - protein, 209 aa, Humulus lupulus
  Humulus lupulus chalcone isomerase
MENNMVMVHEIPFPPEIKTTKPLSLLGYGITDMEIHFLQVKFTAIGVYLDSDVVKHLQQWKGKKGNELAEDDDFFDALISAPVEKYLRIVVIKEIKGSQYGVQLESSVRDRLAAEDMYEEEEEAALEKIVEFLQSKYFKKDTLITFHFPATSPTAEIVVTLEGKEESKLKVENKNVVDMIKKWYLGGTSGVSPSTISSLADNLSAELSK

SEQ ID NO: 4 - protein, 257 aa, Cannabis sativa
  Cannabis sativa (PT1)Truncated
MFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 5 - protein, 313 aa, Mentha x piperita
  geranyl pyrophosphate synthase small subunit [Mentha x piperita]
MAINLSHINSKTCFPLKTRSDLSRSSSGRCMPTAAAAAFPIIATAAQSQPYWAAIEADIDRYLKKSITIRPPETVFGPMHHLTFAAPATAASALCLAACELVGGDRSQAMAAAAAIHLVHAAAYVHEHLPLTDGSRPVSKPAIQHKYGPNVELLTGDGIVPFGFELLAGSVDPARTDDPDRILRVIIEISRAGGPEGMISGLHREEEIVDGNTSLDFIEYVCKKKYGEMHACGAACGAILGGAAEEEIQKLRNFGLYQGTLRGMMEMTNSHQLIDENIIGKLKELALEELGGFHGKNAELMSSLVAEPSLYAA

SEQ ID NO: 6 - protein, 297 aa, Antirrhinum majus
  geranyl diphosphate synthase small subunit [Antirrhinum majus]
MAHGLTHFNTKSGLFPSITKSKTTRPSTRPVILAMTRTQTYRATIESDIESYLKKAIPIRAPESVFEPMHHLTFAAPRTSASALCVAACELVGGDRSDAMAAAAAVHLMHVAAYTHENLPLTDGPMSKSEIQHKFDPNIELLTGDGIIPFGLELMARSMDPTRNNPDRILRAIIELTRVMGSEGIVEGQYHELGLNQLNDLELIEYVCKKKEGTLHACGAACGAILGGCDEDKIEKLRRFGLYVGTVQGLLGKNRSGFEGRIKELKELAVKELESFGGEKIELIRGVFELEHSLAGV

SEQ ID NO: 7 - protein, 279 aa, Humulus lupulus
  geranyl diphosphate synthase small subunit [Humulus lupulus]
MSRTHENHHVPTSTSIVVSASITADIEAHLKQSITLKPPLSVHEPMYNLVFSAPPNSAPSLCVAACELVGGHRSKAIAAASALRLLHAANFTHEHLPLTDSPSPSPVIHNSYDPSIQLLMPDAILPLGFELLAQSYNPAQNNSDRVLRVIVEFARAFGSKGILDGQYRQRVVSISNGDEVDNAERVDCSGREKEGKMHACAAACGAILGDANEEETEKLRTFGLYVGMIQGYSIKFMREREEEKEAERTIKELRNLALKELEHFHGRKLEPISSFIYCL

SEQ ID NO: 8 - protein, 369 aa, Humulus lupulus
  geranyl diphosphate synthase large subunit [Humulus lupulus]
MSSVNLTWVQTCSMFNQAGRSRSSTFNLLHYHPLKKVPFSFQTPKQRRPTSSFSSISAVLTEQEAVTEDEEQKSTFNFKSYMVQKANSVNQALDAAVLLREPQMIHEAMRYSLLAGGKRVRPVLCLSACELVGGDESVAMPAACAVEMIHTMSLIHDDLPCMDNDDLRRGKPTNHKVFGEDVAVLAGDALLAYAFEHVAVSTVGVPAARIIRAIGELAKSIGSEGLVAGQVVDIDSEGLANVGLEQLEFIHLHKTAALLEASVVLGAILGGGTDEQVEELRSFARCIGLLFQAVDDILDVTKSSQELGKTAGKDLVADKATYPRLMGIEKSREFAEKLNREAQEHLGGFDPQKAAPLIALANYIAYRQN

SEQ ID NO: 9 - protein, 382 aa, Abies grandis
  geranyl diphosphate synthase [Abies grandis]
MAYSCMAASCHGLHFMNIASQECNLKRGIIPSKRLHGISSSLWASNGFQGHLERDLSAYRHLVSSSRCLNTIAMLSNLSEQAKEKATEFDFKEYLHSKAISVNEALERAVLRYPEKIHEAMRYSLLAGGKRIRPILTIAACELVGGSEELAMPTACAMEMIHTMSLIHDDLPSMDNDDLRRGKLTNHKVFGEGTAVLAGDALLSFAFEHIAVSTRKTVASHRVLRVVSELGKAIGSQGVAGGQVADITSEGDPSVGLETLEWIHIHKTAVLLECAVVSGAIIGGASENEIERTGRYARCVGLLFQVVDDILDVTRSSEELGKTAGKDLVSDKATYPKLMGLEKAKEFADELLDRAKEELSCFNPAKAAPLLGLADYIALRQN

SEQ ID NO: 10 - protein, 366 aa, Sinapis alba
  geranylgeranyl pyrophosphate synthase [Sinapis alba]
MASSVTPLGSWVLLHHHPSTILTQSRSRSPPSLITLKPISLTPKRTVSSSSSSSLITKEDNNLKSSSSSFDFMSYIIRKADSVNKALDSAVPLREPLKIHEAMRYSLLAGGKRVRPVLCIAACELVGGEESLAMPARCAVEMIHTMSLIHDDLPCMDNDDLRRGKPTNHKVYGEDVAVLAGDALLSFAFEHLASATSSEVSPARVVRAVGELAKAIGTEGLVAGQVVDISSEGLDLNNVGLEHLKFIHLHKTAALLEASAVLGGIIGGGSDEEIERLRKFARCIGLLFQVVDDILDVTKSSQELGKTAGKDLIADKLTYPKLMGLEKSREFAEKLNTEARDQLLGFDSDKVAPLLALANYIANRQN

SEQ ID NO: 11 - protein, 347 aa, Neurospora crassa
  Neurospora crassa OR74A farnesyl-pyrophosphate synthetase (fpp)
MAKTTTLKEFESVFPKLEEALLEYAKAYKLPEQMLSWYKQSLEVNTLGGKCNRGMSVPDSASILLGRPLTEEEYFQAATLGWMTELLQAFFLVSDDIMDSSITRRGKPCWYRQEGVGMVAINDAFMLESAIYTLLKKYFRSHPRYVDFLELFHEVTFQTEMGQLCDLLTAPEDKVDLDNFSMDKYTFIVIYKTAYYSFYLPVALAMYMLDIATPENLKQAEDILIPLGEYFQVQDDYLDNFGLPEHIGKIGTDIQDNKCSWLVNKALSIVTPEQRKTLEENYGRKDKAKEAVIKQLYDDLKLEDHYKQYEEERVGEIRKMIDAIDESKGLKKQVFEAFLGKIYKRSK